(19) United States
(12) Patent Application Publication
(10) Pub. No.: US 2024/0355437 A1
(43) Pub. Date: Oct. 24, 2024

(54) MACHINE LEARNING-BASED SUMMARIZATION AND EVALUATION OF CLINICAL DATA

(71) Applicant: MatrixCare, Inc., Bloomington, MN (US)

(72) Inventors: Jessica ROCKNE, West Jordan, UT (US); Vivek KUMAR, Eden Prairie, MN (US); Kathleen COURSON, Russell, PA (US); Michael DOLCE, Angier, NC (US); Dhawal Bharatbhai KAPADIA, Apex, NC (US)

(21) Appl. No.: 18/618,327

(22) Filed: Mar. 27, 2024

Related U.S. Application Data
(60) Provisional application No. 63/497,551, filed on Apr. 21, 2023.

(51) Int. Cl. G16H 10/60; G06F 40/20; G16H 50/30

(52) U.S. Cl. CPC G16H 10/60; G06F 40/20; G16H 50/30

(57) ABSTRACT

Techniques for machine learning-based data generation are provided. A patient identifier corresponding to a patient is received, and a plurality of patient records is accessed based on the patient identifier. Text data is generated by extracting textual information from the plurality of patient records using one or more text machine learning models. Clinical data is generated by processing the text data using one or more extraction machine learning models, and summarized clinical data is generated by processing the clinical data using one or more summary machine learning models. The summarized clinical data is output.

### CROSS-REFERENCE TO RELATED APPLICATIONS

This application claims priority to U.S. Provisional Patent Application No. 63/497,551, filed Apr. 21, 2023, the entire content of which is incorporated herein by reference in its entirety.

### INTRODUCTION

Aspects of the present disclosure relate to machine learning. More specifically, aspects of the present disclosure relate to training and using machine learning to summarize clinical data and generate various risk measures and/or care plans.

In a wide variety of healthcare settings, voluminous records are generated and maintained to track or reflect the current and/or prior health of patients. For example, records such as physician's notes, images, audio and/video recordings, lab reports, and the like are often generated at various points in a patient's path (including in connection with interactions directly with the patient, such as during an appointment, as well as in connection with non-interaction events, such as if the patient suffers an injury). In many cases, the sheer volume of documents renders human review impractical or simply impossible. Further, in many instances, the documents (which are often unstructured) contain contradictory information, duplicative information, and the like, rendering them further inaccessible to conventional systems.

These data concerns can be particularly problematic when a patient transfers to a different facility or enterprise. For example, the new enterprise may wish to understand the patient's history, but conventional systems are generally limited to manually scanning or entering information (in the case of physical records) and/or manually transmitting the information (e.g., via fax, email, online portal, and the like) to the new enterprise. This is expensive, error-prone, and manually-intensive. Further, the large number of records for many patients renders such a process impossible over realistic time scales (e.g., limiting data to only the last year, for example). Moreover, the receiving entity is generally incapable of effective review and understanding of the data, owing to the above-discussed problems.

Improved systems and techniques to summarize clinical data and predict patient risks are desired.

### SUMMARY

According to one embodiment presented in this disclosure, a method is provided. The method includes: receiving a patient identifier corresponding to a patient; accessing a plurality of patient records based on the patient identifier; generating text data by extracting textual information from the plurality of patient records using one or more text machine learning models; generating clinical data by processing the text data using one or more extraction machine learning models; generating summarized clinical data by processing the clinical data using one or more summary machine learning models; and outputting the summarized clinical data.

According to a second embodiment of the present disclosure, a method is provided. The method includes: accessing a plurality of patient records of a patient; generating text data by extracting textual information from the plurality of patient records using one or more text machine learning models; generating clinical data by processing the text data using one or more extraction machine learning models; generating summarized clinical data by processing the clinical data using one or more summary machine learning models; and training one or more risk prediction machine learning models to generate predicted risk measures based on the summarized clinical data.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for using machine learning to summarize and evaluate patient records, according to one embodiment of the present disclosure.

FIG. 2 depicts an example workflow for generating summarized clinical data, according to one embodiment of the present disclosure.

FIG. 3 depicts an example workflow for generating summarized clinical data using machine learning, according to one embodiment of the present disclosure.

FIG. 4 depicts an example workflow for summarizing clinical data using machine learning, according to one embodiment of the present disclosure.

FIG. 5 depicts an example workflow for preprocessing unstructured data for improved machine learning.

FIG. 6 depicts an example workflow for training machine learning models based on summarized clinical data, according to one embodiment of the present disclosure.

FIG. 7 depicts an example workflow for using machine learning models to evaluate summarized clinical data, according to one embodiment of the present disclosure.

FIG. 8 is a flow diagram depicting an example method for generating and evaluating summarized clinical data using machine learning models, according to one embodiment of the present disclosure.

FIG. 9 is a flow diagram depicting an example method for generating summarized clinical data using machine learning, according to one embodiment of the present disclosure.

FIG. 10 is a flow diagram depicting an example method for generating clinical data based on extracted text, according to one embodiment of the present disclosure.

FIG. 11 is a flow diagram depicting an example method for generating summarized clinical data, according to one embodiment of the present disclosure.

FIG. 12 is a flow diagram depicting an example method for preprocessing unstructured input data to improve machine learning results, according to one embodiment of the present disclosure.

FIG. 13 is a flow diagram depicting an example method for training machine learning models to evaluate summarized clinical data, according to one embodiment of the present disclosure.

FIG. 14 is a flow diagram depicting an example method for using machine learning models to evaluate summarized clinical data, according to one embodiment of the present disclosure.

FIG. 15 is a flow diagram depicting an example method for generating and outputting summarized clinical data, according to one embodiment of the present disclosure.

FIG. 16 is a flow diagram depicting an example method for training machine learning models based on summarized clinical data, according to one embodiment of the present disclosure.

FIG. 17 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved clinical data summarization and risk prediction using machine learning.

In some embodiments, patient records may be evaluated using various machine learning models and techniques to generate summarized clinical data representing the relevant or salient health-related or medical-related information for a patient with respect to a desired outcome or event. That is, in some embodiments, the particular content and format of the summarized data may vary depending not only on the particular implementation, but also on the intended use or purpose of the data. For example, the machine learning-based techniques described herein may be used to generate different sets of summarized clinical data for different purposes, such as one set for specialist referral purposes (e.g., when a patient is referred to a specialist) and a second set for discharge purposes (e.g., when a patient is discharged from a hospital). In some examples discussed herein, clinical data is summarized to facilitate provisioning of home health services (e.g., when a physician or clinician recommends that the patient receive medical or healthcare-related services in their home). However, aspects of the present disclosure are readily applicable to a wide variety of healthcare (and non-healthcare) related implementations.

In some embodiments, to generate summarized clinical data, a sequence of machine learning (ML) components may be used. For example, given a set of patient records (e.g., written documents, notes, lab reports, and the like), a first ML component may be used to identify and extract textual data. A second ML component may then be used to extract relevant concepts and information from the text, and a third ML component may be used to generate summaries for this extracted information.

In some embodiments, the summarized clinical data may be output for review or use by users or downstream systems. In some embodiments, the summarized information is output for display (e.g., via a graphical user interface (GUI)) to a user (e.g., a clinician or physician). For example, in some embodiments, the summarized data may be used to generate a visual report (e.g., a brief document having the salient or relevant information) summarizing the patient's information. As one example, when a physician refers a patient for home health services, the home health service agency may request patient records for the patient. In some aspects, these records may be voluminous and unstructured, rendering manual review impossible. In an embodiment, therefore, the home health service may utilize aspects disclosed herein to summarize the relevant features of the patient data, enabling rapid manual (or automated) review.

In some embodiments, in addition to summarizing clinical data, the system can further generate and/or embed reference indications in the summary document to indicate the source of each portion of the summarized data. For example, for one portion or element in the summarized data (e.g., a diagnosis), the system may generate an indication that the diagnosis is disclosed or reflected in a given document (e.g., indicating a unique identifier of the document). In some embodiments, the reference indication may include additional information such as the name of the document, the physician (if any) associated with the document (e.g., the doctor that wrote the note), the date of the document's creation, the specific portion or point in the document that includes the information, and the like. In some embodiments, the reference indications are embedded in the summary document (e.g., as footnotes, or placed next to the relevant portion or element that they provide support for). In some embodiments, the reference indications can include pointers or links to the support documentation/to specific portions of the documentation, allowing users to readily access the specific supporting information for any element of the summarized data.

In some embodiments, in addition to or instead of outputting the summarized data via a GUI or other means, the system may output the summarized data as input to another component or system (or to a process on the same system), such as a machine learning model. For example, the summarized clinical data may be used as input to one or more risk prediction models trained to predict various risks to patients (e.g., a fall risk, a medication risk, a re-hospitalization risk, and the like), as discussed in more detail below. As another example, the summarized clinical data and/or predicted risks (generated using one or more risk ML models) can be used as input to a care plan generation ML model trained to generate recommended care plans for patients, as discussed in more detail below.

Advantageously, by using automatically-generated summarized information to train the machine learning models, model training can be realistically and practically performed for a wider variety of solutions. For example, because machine learning models often rely on massive amounts of training data to function accurately, it is often impractical or impossible to train models for desired purposes because the needed training data simply does not exist. In some embodiments of the present disclosure, this training data can be generated efficiently and reliably, thereby enabling expanded training and use of machine learning. Further, by using automatically generated summarized information as input during runtime, aspects of the present disclosure enable machine learning to be used in vastly more instances as compared to conventional approaches. For example, as discussed above, the data associated with a patient may be unstructured and voluminous, such that there is no clear input that can be used with ML. By generating summarized clinical data, however, embodiments of the present disclosure enable efficient, rapid, and accurate ML-based predictions to be generated for patients with little or no manual effort.

### Example Environment for Using Machine Learning to Summarize and Evaluate Patient Records

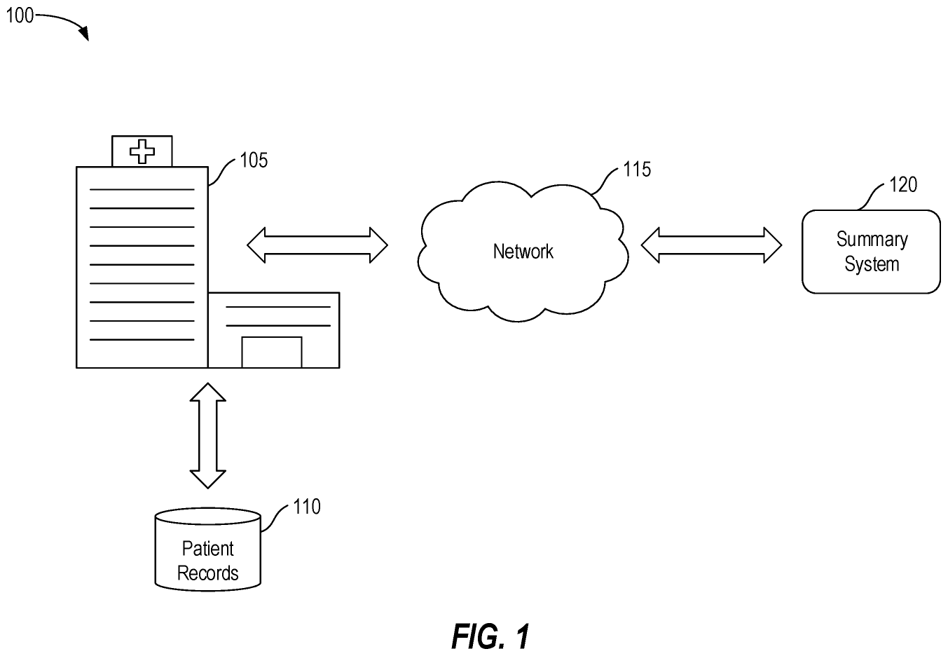

FIG. 1 depicts an example environment 100 for using machine learning to summarize and evaluate patient records, according to one embodiment of the present disclosure.

In the illustrated example, a hospital 105 having a set of patient records 110 is communicatively coupled with a summary system 120 via a network 115. The network 115 can generally include one or more networks or other communication links, including wired links, wireless links, or a combination of wired and wireless links. In at least one embodiment, the network 115 corresponds to or includes the Internet. Although a discrete summary system 120 is depicted for conceptual clarity, in some embodiments, the summary system 120 may be implemented as a component of another system, and may generally be implemented using hardware, software, or a combination of hardware and software. Additionally, though depicted as a discrete system separate from the hospital 105, in some aspects, the summary system 120 is implemented by or in the hospital 105.

Although the illustrated example depicts a hospital 105 for conceptual clarity, in embodiments, a wide variety of entities or institutions may maintain the patient records 110. For example, the hospital 105 may be a clinic, doctor's office, or other record-maintaining entity. In some embodiments, the hospital 105 may correspond to any healthcare-related entity that stores or provides access to patient records 110. Additionally, although healthcare-related entities are discussed in some examples of the present disclosure, the embodiments disclosed herein are readily applicable to a wide variety of other (non-healthcare related) implementations.

The patient records 110 generally include information or data for one or more patients. As used herein, a “patient” can generally include any individual who has received, is currently receiving, or will receive one or more healthcare-related services. In some aspects, “patients” may additionally or alternatively be referred to as “users.” The patient records 110 generally comprise healthcare-related information for the one or more patients, such as lab reports, free form (e.g., natural language) notes authored by healthcare providers (e.g., physicians or clinicians), and the like. In some aspects, the patient records 110 may include continuity of care documents (CCDs), which is generally a summary of a patient's clinical information used to facilitate continued or new care (e.g., when the patient is transferred to a different facility). As additional (non-limiting) examples, the patient records 110 may include other data such as audio, video, and/or image data of the patient(s) and/or of another user discussing or interacting with the patient (e.g., a video of a doctor speaking with the patient, an audio recording of the doctor's discussing his thoughts after a visit with a patient, and the like).

In some embodiments, the patient records 110 may generally include electronic records (e.g., electronic health records (EHR)), physical records (e.g., physical documents in a filing system), or a combination of the physical and electronic documents.

In some embodiments, the summary system 120 can use one or more machine learning techniques to generate summarized clinical information for patients, as discussed in more detail below. In some embodiments, the summary system 120 may additionally or alternatively use one or more machine learning techniques to evaluate summarized clinical information, such as to predict various risks, to generate recommended care plans, and the like, as discussed in more detail below.

In at least one embodiment, the summary system 120 may access the patient record(s) 110 of a given patient by requesting them from the hospital 105. As used herein, “accessing” data can generally include receiving, requesting, retrieving, requesting, obtaining, or otherwise gaining access to the data. For example, upon receiving a request or referral indicating a specific patient (e.g., using a specific patient identifier), the summary system 120 may transmit, to the hospital 105 (or to any other potential holders of the patient information), a request for patient records 110 pertaining to or specifying the specific patient/identifier. As one example, if a clinician at the hospital 105 has provided a referral (e.g., for home health services), the summary system 120 may transmit the request to the hospital 105 and/or clinician that initiated or provided the referral. In the illustrated example, the summary system 120 can thereby receive or otherwise access the relevant patient records 110 for the patient.

In this way, the summary system 120 can efficiently access the patient records and use machine learning to generate and/or evaluate clinical summaries for the patient, thereby substantially improving patient outcomes, such as by reducing mistakes which can cause harm to the patient, and/or by enhancing or enabling services to be provided more rapidly and accurately, thereby improving benefits to the patient. Further, by training and/or using machine learning to evaluate the documentation, the summary system 120 can improve accuracy while reducing computational expense (e.g., storage, memory, and/or processor requirements), as well as reducing manual effort.

### Example Workflow for Generating Summarized Clinical Data

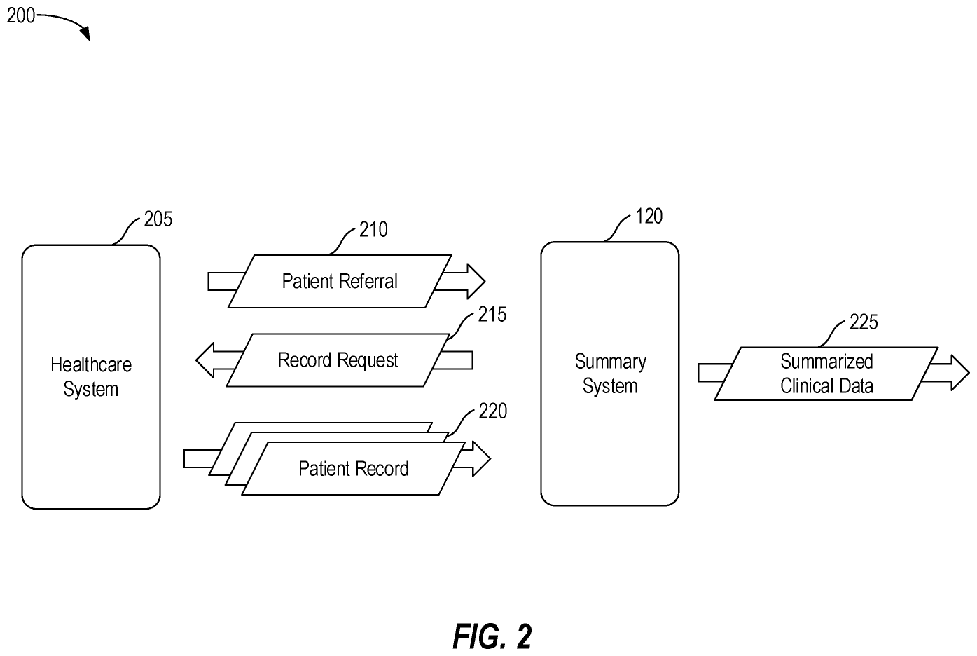

FIG. 2 depicts an example workflow 200 for generating summarized clinical data, according to one embodiment of the present disclosure. In some embodiments, some or all of the workflow 200 may be performed within or for the environment 100 of FIG. 1.

In the illustrated example, a healthcare system 205 is communicatively coupled with a summary system 120. Though depicted as a discrete system for conceptual clarity, in some embodiments, the healthcare system 205 may be implemented as a component of another system, and may generally be implemented using hardware, software, or a combination of hardware and software. In at least one embodiment, the healthcare system 205 is a computational system of a healthcare facility, such as a hospital (e.g., the hospital 105 of FIG. 1), a clinic, and the like. Generally, the healthcare system 205 may correspond to any system that stores, maintains, or otherwise provides access to patient records.

In the illustrated workflow 200, the healthcare system 205 transmits a patient referral 210 to the summary system 120. Generally, a referral may comprise an order, instruction, or recommendation by a healthcare provider that a specific patient should pursue and/or receive healthcare services (either specifically enumerated services, or broad service categories) from a recipient of the referral. For example, a doctor may author and provide a patient referral 210 to a home healthcare service entity (e.g., a facility or entity that provides or manages home healthcare services) indicating that the patient would benefit from or should receive one or more healthcare services in their home. As additional examples, the patient referral 210 may indicate that the patient needs or should receive specialist care, additional follow-up with another facility (e.g., to capture additional imaging), and the like.

In the illustrated example, the summary system 120 receives the patient referral 210. In some aspects, rather than receiving the referral, the summary system 120 may receive a summary request. That is, in some embodiments, the summary system 120 may be associated with the facility or entity that receives the patient referral 210 (e.g., the summary system 120 may be maintained by a home health services entity). In other embodiments, the summary system 120 may be an independent system which can be accessed or used by other entities. For example, when a home health service provider receives a patient referral 210, the service provider may transmit the referral and/or a request (identifying the specific patient and/or the referring healthcare system 205) for a clinical summary to the summary system 120.

In the illustrated example, the summary system 120 then transmits, to the healthcare system 205, a record request 215. In some embodiments, the summary system 120 transmits the record request 215 to the entity that provided the patient referral 210. For example, if the patient referral 210 was received directly by the summary system 120, the summary system 120 may respond with the record request 215. If the patient referral 210 was transmitted to a third entity that requested a clinical data summary from the summary system 120, the third entity may indicate the origin of the referral and/or the entity that has (or may have) records, and the summary system 120 may transmit the record request 215 to the identified entity (or entities).

In some embodiments, the patient referral 210 can include a patient identifier of the patient, which may comprise one or more elements. For example, the patient identifier may include a unique identifier (e.g., a string of numbers and/or letters) assigned by the healthcare system 205, or may include one or more other elements of information used to identify the patient (e.g., their name, birth date, social security number, address, and the like). In the illustrated example, the summary system 120 may include this identifier information in the record request 215, allowing the healthcare system 205 to efficiently identify relevant records associated with the specific patient.

In the illustrated workflow 200, the healthcare system 205 can then identify one or more relevant patient records 220 based on the patient identifier, and provide the records to the summary system 120. In embodiments, as discussed above, the patient records 220 can generally include a wide variety of healthcare information for the patient, such as lab reports, physician's notes, and the like. In some embodiments, the patient records 220 can include one or more electronic records which may be transmitted electronically (e.g., via email or some other electronic method of delivery) and/or one or more physical records (which may be transmitted physically, such as by mail or courier, may be scanned to enable electronic delivery, and/or may be faxed). In some embodiments, the patient records 220 can include one or more CCDs, which may be prepared by the healthcare system 205 in response to the record request 215, or may have been pre-prepared (e.g., during a prior transfer of the patient).

Generally, the patient records 220 can include any number and variety of records (also referred to as documents in some aspects) formatted according to any number and variety of formats, including image data, textual data, audio data, video data, and the like. Further, a single record or document may include data of multiple types or formats, such as image data and textual data. In some embodiments, textual data may be specifically formatted/identified as text (e.g., using ASCII and/or Unicode characters). In some embodiments, textual data may be included in other data (e.g., image or video data) without specific electronic identification of the characters therein. In some embodiments, textual data may take the form of verbal or spoken text (e.g., in a recording of a doctor talking about a patient).

In the illustrated example, the summary system 120 accesses and evaluates the patient records 220 to generate summarized clinical data 225. In some embodiments, the summary system 120 uses one or more machine learning models or techniques to generate the summarized clinical data 225, as discussed in more detail below. For example, the summary system 120 may use a first ML component to identify, extract, and/or generate textual data based on the patient records 220. A second ML component may then be used to identify, extract, or otherwise recognize one or more relevant concepts (e.g., clinical data) from the textual data, as discussed in more detail below. In an embodiment, a third ML component may then be used to summarize the identified clinical data, such as by eliminating duplicative information, consolidating or grouping contextually similar or relevant information, and the like.

In some embodiments, as discussed above, the summary system 120 may output the summarized clinical data 225 via various means, including display via a GUI (e.g., to a user), transmission to another system or component (e.g., a machine learning system that uses the summarized clinical data 225 as model input), and the like. In at least one embodiment, as part of generating the summarized clinical data 225, the summary system 120 can additionally generate reference indications indicating, for each element or portion of the summarized clinical data 225, the specific source or support for the element. For example, for an element corresponding to the patient's age, the summary system 120 may generate a reference indication pointing to one or more specific patient records 220 that indicate the patient's date of birth. Similarly, for an element corresponding to a diagnosis of a specific disorder, the summary system 120 may generate a reference indication pointing to one or more patient records 220 that indicate diagnosis of the disorder (e.g., in a doctor's note).

In some embodiments, the summary system 120 can store the summarized clinical data 225 locally or remotely, and/or may provide the summarized clinical data 225 to one or more other systems, such as providing it to the healthcare system 205, transmitting it to an entity that requested the summary, and the like.

### Example Workflow for Generating Summarized Clinical Data Using Machine Learning

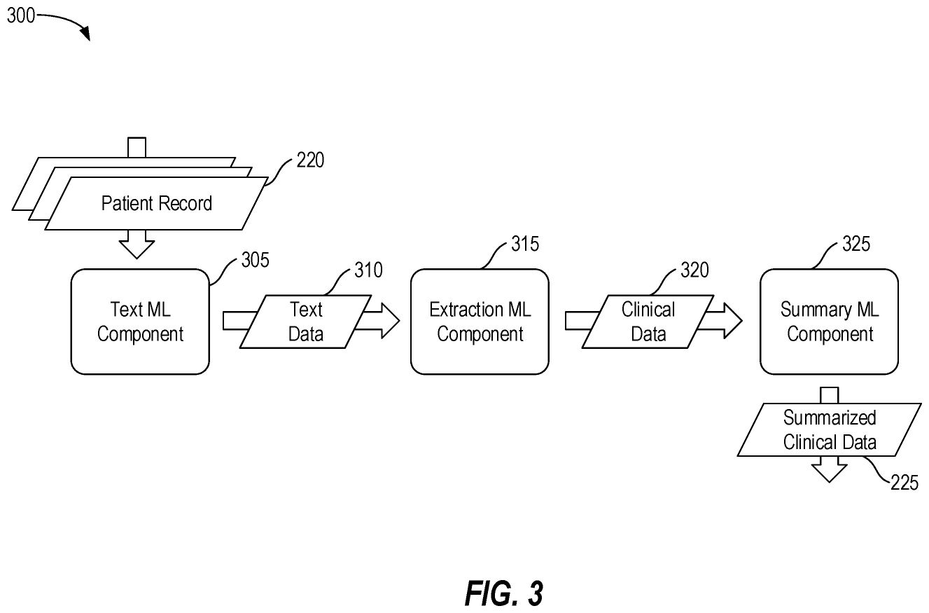

FIG. 3 depicts an example workflow 300 for generating summarized clinical data using machine learning, according to one embodiment of the present disclosure. In some embodiments, the workflow 300 is performed by a summary system, such as the summary system 120 of FIGS. 1 and/or 2. That is, the depicted components may be components of the summary system 120.

Although a discrete text ML component 305, extraction ML component 315, and summary ML component 325 are depicted for conceptual clarity, in some embodiments, the operations of the depicted components (and others not depicted in the illustrated example) may be combined or distributed across any number and variety of components and systems. Generally, the operations of the depicted components may be implemented using hardware, software, or a combination of hardware and software.

In the illustrated example, patient records 220 are accessed and evaluated by a text ML component 305 (e.g., using a text machine learning model) to generate text data 310. Generally, the text ML component 305 may use one or more ML-based techniques or models to extract text information from the patient records 220. For example, the text ML component 305 may identify and extract any text that is included using Unicode or ASCII format. In some embodiments, the text ML component 305 may use optical character recognition (OCR) or other ML-based models to identify and extract/generate textual information from images included in the patient records 220 (e.g., from a scanned image of handwritten text). In some embodiments, the text ML component 305 may use one or more voice-to-text ML models to generate textual information based on spoken or verbal natural language (e.g., from audio and/or video recordings in the patient records 220). In this way, the text data 310 can generally include computer-readable text that was identified in, extracted from, or otherwise generated based on the patient records. In at least one embodiment, while extracting or generating the text data 310, the text ML component 305 may generate and include reference indications for each element of the text data 310 to indicate its respective source (e.g., to indicate the specific patient record(s) 220 used to generate the element of text data, the specific location(s) in the record(s), and the like). For example, the text ML component 305 may embed the reference information as metadata with the text data 310.

In the illustrated example, the text data 310 is accessed and evaluated by an extraction ML component 315 (e.g., using an extraction machine learning model) to generate clinical data 320. Generally, the extraction ML component 315 may use one or more ML-based techniques or models to identify and extract clinical information from the text data 310. For example, the extraction ML component 315 may use keyword searching and/or machine learning model-based evaluations, such as processing the text data 310 using one or more large language models, one or more prompt models, and the like. In some embodiments, the specific concepts, features, or information extracted to generate the clinical data 320 may vary depending on the particular implementation. In at least one embodiment, the extraction ML component 315 extracts any patient demographic information (e.g., age, gender, race, height, weight, and the like), home treatment information (e.g., indicating which home services, if any, are currently received and/or recommended), therapy information (e.g., pertaining to or indicating therapies that the patient engages in, diagnosis information (e.g., indicating diagnoses the patient has received, when each diagnosis was received or documented, and the like), medication information (e.g., indicating medications that the patient currently takes and/or previously took), and the like. In at least one embodiment, while extracting or generating the clinical data 320, the extraction ML component 315 may generate and include reference indications (or preserve reference indications generated by the text ML component 305) for each element of the clinical data 320 to indicate its respective source (e.g., to indicate the specific patient record(s) 220 used to generate the element of clinical data, the specific location(s) in the record(s), and the like). For example, the extraction ML component 315 may embed the reference information as metadata with the clinical data 320.

In some embodiments, the extraction ML component 315 can identify text data 310 pertaining to face-to-face meetings between the patient and a physician, and extract relevant information from each, such as the date when the meeting occurred, any certifications or indications of the physician (e.g., certifying that the patient would benefit from home health services), relevant explanations for the physician's certification or opinion (e.g., explaining why the patient would benefit from home health services), indications as to any specific services that are recommended, and the like.

For example, in some embodiments, prior to approving or receiving home health services, the patient (or physician) may review various elements of clinical information, such as to confirm that the patient is homebound (e.g., due to a general inability to leave the home, and/or if leaving the home requires considerable and taxing effort), to confirm that the patient actually needs or would benefit from home services (e.g., if because of illness or injury, the patient needs the aid of supportive devices such as crutches, canes, wheelchairs, and walkers, the use of special transportation, and/or the assistance of another person, or if the patient has a condition such that leaving their home is medically contraindicated). In an embodiment, such information can be identified and extracted, by the extraction ML component 315, from the text data 310.

In the illustrated example, the clinical data 320 is accessed and evaluated by a summary ML component 325 (e.g., using a summary machine learning model) to generate summarized clinical data 225. Generally, the summary ML component 325 may use one or more ML-based techniques or models to summarize the extracted clinical data 320. For example, the summary ML component 325 may use keyword searching and/or machine learning model-based evaluations, such as processing the clinical data 320 using one or more large language models, one or more prompt models, and the like. In some embodiments, the summary ML component 325 may use various ML and/or keyword-based techniques to group information based on context.

That is, in one embodiment, the summary ML component 325 may group, cluster, or otherwise associate various elements of the clinical data 320 into groups based on the underlying context of each element of data. For example, the summary ML component 325 may identify any clinical data 320 relevant to a specific diagnosis, and group this data under that diagnosis. Similarly, the summary ML component 325 may group data associated with a given face-to-face meeting with a physician. In some embodiments, a given element of clinical data 320 may be present in or associated with multiple groups.

In some embodiments, the summary ML component 325 may additionally or alternatively evaluate the clinical data 320 to eliminate redundant or duplicative information. For example, if the same diagnosis is reflected in multiple records, the clinical data 320 may include multiple instances of this diagnosis. In some embodiments, the summary ML component 325 may eliminate or remove this duplicative information such that the summarized clinical data 225 includes a single instance of it. In some embodiments, removing duplicative information can include removing information that is duplicative within a given group of the clinical information, and/or removing information that is duplicative globally (across all defined groups).

In at least one embodiment, while generating the summarized clinical data 225, the summary ML component 325 may generate and include reference indications (or preserve reference indications generated by the text ML component 305 and/or extraction ML component 315) for each element of the summarized clinical data 225 to indicate its respective source (e.g., to indicate the specific patient record(s) 220 used to generate the element of summarized clinical data, the specific location(s) in the record(s), and the like). For example, the summary ML component 325 may embed the reference information as metadata with the summarized clinical data 225. In some embodiments, the summary ML component 325 may additionally or alternatively embed or add the reference information by using footnotes and/or endnotes in the summarized clinical data 225 (e.g., if the summarized clinical data 225 is textual in form), by inserting the reference indications themselves into the text of the summarized clinical data 225 (e.g., next to or below the corresponding element), and the like.

In this way, using the workflow 200, the depicted components can readily and efficiently generate accurate and reliable summarized clinical data 225 with minimal (or no) manual effort. Advantageously, by reviewing such summarized clinical data 225 (e.g., manually reviewing the summarized data rather than the raw data), the effort, time, and potential for error is substantially reduced. Further, if the summarized clinical data 225 is used as input to one or more downstream systems (e.g., machine learning models), the overall computational efficiency is reduced substantially (e.g., because evaluating the summarized data consumes substantially fewer resources, such as power, compute time, memory, and the like, as compared to evaluating the raw data).

### Example Workflow for Preprocessing Unstructured Data for Improved Machine Learning

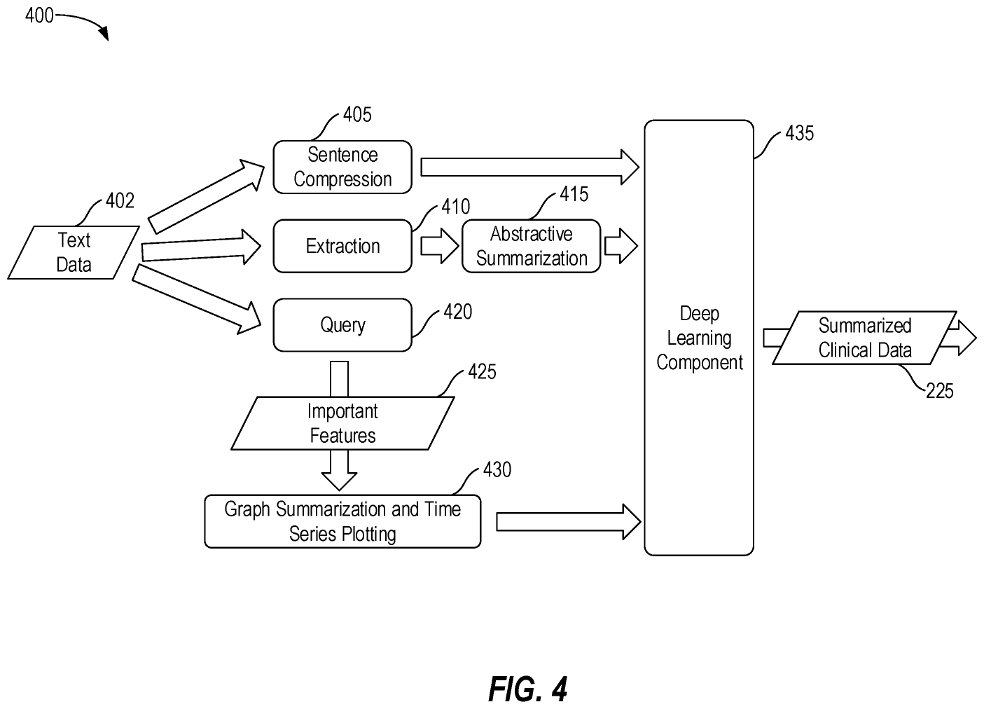

FIG. 4 depicts an example workflow 400 for summarizing clinical data using machine learning, according to one embodiment of the present disclosure. In some embodiments, the workflow 400 is performed by one or more components of a summary system, such as the summary system 120 of FIG. 2. For example, the workflow 400 may be performed by the summary ML component 325 of FIG. 3 and/or the extraction ML component 315 of FIG. 3.

In the illustrated example, text data 402 is accessed and processed according to a variety of operations. In some embodiments, the text data 402 may correspond to the text data 310 of FIG. 3, and/or to the clinical data 320 of FIG. 3. In some embodiments, the text data 402 generally corresponds to raw text extracted from patient documents/records.

In the illustrated embodiment, operation 405 (labeled “sentence compression”) may generally correspond to compressing one or more sentences in the text data 402 using one or more compression techniques or criteria. For example, in some embodiments, operation 405 may include deleting or removing one or more words (e.g., deleting redundant and/or superfluous words that do not substantively add to the meaning of the text), inserting one or more words (if needed for clarity or to replace deleted words), reordering words to improve clarity (e.g., if deleting one or more words affects clarity), and the like. As illustrated, the output of the operation 405 (e.g., compressed versions of the sentences in the text data 402) are provided to a deep learning component 435, discussed in more detail below.

In the illustrated example, operation 410 (labeled “extraction”) may generally correspond to the extraction of important sentences, keywords, phrases, and the like from the text data 402. In some aspects, “importance” of a given portion of the text data 402 may be determined according to a variety of criteria, including its location in the document(s) from which it was extracted, any metadata associated with that location and/or with the text itself and/or the document itself, determining relevance of the text with respect to one or more defined keywords or phrases, and/or scoring the sentences, words, and/or phrases using one or more techniques or models (e.g., machine learning models, natural language processing (NLP) models, and the like) to assign numerical scores to each based on relative importance.

In the illustrated example, the output of the operation 410 (e.g., extracted important sentences, words, and/or phases) are processed by operation 415 (labeled “abstractive summarization”), which may generally correspond to using one or more NLP techniques to generate a short/concise summary capturing the salient details of the text. In some embodiments, the resulting text may generally be shorter than the input text, and may include text (e.g., phrases or sentences) that were not included in the original text. As illustrated, the output of the operation 415 is provided to a deep learning component 435, discussed in more detail below.

In the illustrated example, operation 420 (labeled “query”) may generally correspond to the query-based extraction of important features from the text data 402. In some aspects, the important attributes for which features are extracted (using various queries) may include, without limitation, medication and administration information, diagnoses, vitals, therapy information, treatment information, and the like. As illustrated, the resulting important features 425 are then provided to an operation 430 (labeled “graph summarization and time series plotting”) for processing.

In an embodiment, the operation 430 may generally include a variety of techniques and operations. For example, the operation 430 may include use of a graph-based approach to text summarization, using an unsupervised machine learning technique to rank or score sentences or words based on a graph. That is, the text can be graphed and the graph can be evaluated to determine or infer the importance of any given node (e.g., sentence or phrase) in the important features 425. As another example, the operation 430 may include use of time series plotting of any data (in the important features 425) that changes over time (e.g., indicating when various medications were used, vitals at different times, and the like). As illustrated, the output of the operation 415 is provided to a deep learning component 435, discussed in more detail below.

In the illustrated workflow 400, the outputs of the operations 405, 415, and 430 are provided as input to a deep learning component 435. The deep learning component 435 may generally use a variety of techniques or operations to generate summarized clinical data 225 based on its inputs. For example, in at least one embodiment, the deep learning component 435 may use one or more machine learning models or algorithms to evaluate the data at various levels (e.g., a neural network having multiple layers) to extract information at various degrees of granularity/specificity. In the illustrated example, the output of the deep learning component 435 is summarized clinical data 225. As discussed above, this may generally be formatted in a variety of ways, including as textual data (e.g., as a text document arranged in a human-readable form), as key-value data (e.g., with relevant features for each key or field), and the like.

### Example Workflow for Preprocessing Unstructured Data for Improved Machine Learning

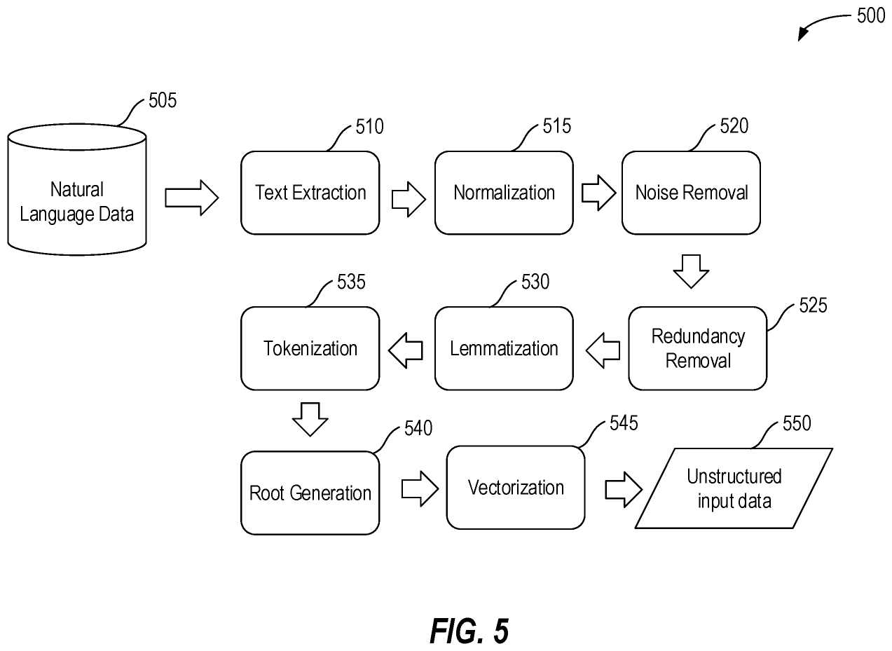

FIG. 5 depicts an example workflow 500 for preprocessing unstructured data for improved machine learning.

In some embodiments, the workflow 500 may be performed to process natural language data 505 for input to one or more machine learning models. In some embodiments, the workflow 500 is performed by one or more remote systems (e.g., by a cloud-based service). In other embodiments, the workflow 500 is performed by a machine learning system or summary system, such as summary system 120 of FIGS. 1 and 2. The workflow 500 may be used during training of machine learning models (e.g., to generate the training input) and/or during inferencing using the models. That is, the workflow 500 may be used to transform or preprocess any natural language input, prior to it being used as input to the machine learning model(s) during training or inferencing.

In some embodiments, the various depicted operations may be performed by one or more components of the summary system 120 (e.g., by the text ML component 305, extraction ML component 315, and/or summary ML component 325, each of FIG. 3) as part of their processing, and/or to prepare the data for evaluating using their respective ML models.

In the illustrated workflow 500, natural language data 505 is accessed for processing to generate unstructured input data 550. In some embodiments, the workflow 500 is referred to as preprocessing to indicate that it is used to transform, refine, manage, or otherwise modify the natural language data 505 to improve its suitability for use with machine learning systems (or other downstream processing). In some embodiments, the natural language data 505 corresponds to or is embedded in patient data (e.g., patient records).

In some embodiments, preprocessing the data in the natural language data 505 may improve the ML training process by making the data more compatible with natural language processing, and ultimately for consumption by the ML model during training. Preprocessing can generally include a variety operations. Though the illustrated workflow 500 depicts a series of operations being performed sequentially for conceptual understanding, in embodiments, some or all of the operations may be performed in parallel. Similarly, in embodiments, the workflow 500 may include additional operations not depicted, or may include a subset of the depicted operations.

In the illustrated example, the natural language data 505 can first undergo text extraction 510. The text extraction 510 generally corresponds to extracting natural language text from an unstructured portion of the natural language data 505. For example, if the natural language data 505 includes a set of progress notes (e.g., notes written by a clinician describing an encounter with a user or patient), the text extraction 510 can include identifying and extracting these notes for evaluation. In some aspects, the notes may further include structured or semi-structured data that can undergo more traditional processing as needed, such as a timestamp indicating when the note was written or revised, an indication of the specific patient about whom the note was written, the author of the note, and the like.

The normalization 515 can generally a wide variety of text normalization processes, such as converting all characters in the extracted text to lowercase, converting accented or foreign language characters to ASCII characters, expanding contractions, converting words to numeric form where applicable, converting dates to a standard date format, and the like.

Noise removal 520 can generally include identification and removal of portions of the extracted text that do not carry meaningful or probative value. That is, noise removal 520 may include removing characters, portions, or elements of the text that are not useful or meaningful in the ultimate computing task (e.g., computing a predicted efficacy score), and/or that are not useful to human readers. For example, the noise removal 520 may include removing extra white or blank spaces, tabs, or lines, removing tags such as HTML tags, and the like.

Redundancy removal 525 may generally correspond to identifying and eliminating or removing text corresponding to redundant elements (e.g., duplicate words), and/or the reduction of a sentence or phrase to a portion thereof that is most suitable for machine learning training or application. For example, the redundancy removal 525 may include eliminating verbs (which may be unhelpful in the machine learning task), conjunctions, or other extraneous words that do not aid the machine learning task.

Lemmatization 530 can generally include stemming and/or lemmatization of one or more words in the extracted text. This may include converting words from their inflectional or other form to a base form. For example, lemmatization 530 may include replacing “holding,” “holds,” and “held” with the base form “hold.”

In one embodiment, tokenization 535 includes transforming or splitting elements in the extracted text (e.g., strings of characters) into smaller elements, also referred to as “tokens.” For example, the tokenization 535 may include tokenizing a paragraph into a set of sentences, tokenizing a sentence into a set of words, transforming a word into a set of characters, and the like. In some embodiments, tokenization 535 can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

In an embodiment, root generation 540 can include reducing portion of the extracted text (e.g., a phrase or sentence) to its most relevant n-gram (e.g., a bigram) or root for downstream machine learning training and/or application.

Vectorization 545 may generally include converting the text into one or more objects that can be represented numerically (e.g., into a vector or tensor form). For example, the vectorization 545 may use one-hot encodings (e.g., where each element in the vector indicates the presence or absence of a given word, phrase, sentiment, or other concept, based on the value of the element). In some embodiments, the vectorization 545 can correspond to any word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate efficacy scores).

As illustrated, the various preprocessing operations in the workflow 500 result in generation of unstructured input data 550. That is, the unstructured input data 550 corresponds to unstructured natural language data 505 that has undergone various preprocessing to improve its use with downstream machine learning models. The preprocessing workflow 500 can generally include any other suitable techniques for making text ingestion more efficient or accurate (either in a training phase of a machine learning model, or while generating an inference or prediction using a trained model). Generally, improving the results of this natural language processing can have significant positive impacts on the computational efficiency of processing the data downstream, as well as the eventual accuracy of the trained machine learning model(s).

In some embodiments, as discussed above, this unstructured input data 550 corresponds to summarized clinical data 225 of FIGS. 2-3, and may be output for review and/or used as input to one or more machine learning models, as discussed in more detail below.

### Example Workflow for Training Machine Learning Models Based on Summarized Clinical Data

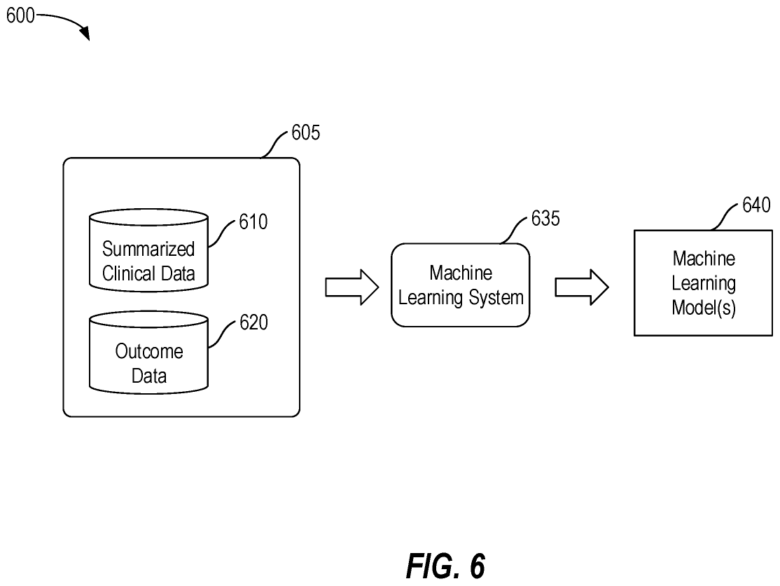

FIG. 6 depicts an example workflow 600 for training machine learning models based on summarized clinical data, according to one embodiment of the present disclosure.

In the illustrated workflow 600, a set of training data 605 (also referred to as patient data, user data, resident data, or historical data in some embodiments) is evaluated by a machine learning system 635 to generate or train one or more machine learning models 640. In embodiments, the machine learning system 635 may be implemented using hardware, software, or a combination of hardware and software. In some embodiments, the machine learning system 635 corresponds to the summary system 120 of FIGS. 1-2.

The training data 605 generally includes data or information associated with one or more patients (also referred to as users or residents (such as in a long-term residential care facility) in some aspects) from one or more prior points in time. That is, the training data 605 may include, for one or more patients, a set of one or more snapshots of the resident's characteristics or attributes (reflected by summarized clinical data 610) at one or more points in time. In some embodiments, the training data 605 includes information for patients of one or more healthcare facilities or professionals, residents residing in one or more long-term care facilities, and the like. The training data 605 may generally be stored in any suitable location. For example, the training data 605 may be stored within the machine learning system 635, or may be stored in one or more remote repositories, such as in a cloud storage system.

In the illustrated example, the training data 605 includes, for each exemplar reflected in the data, a set of summarized clinical data 610 and corresponding outcome data 620. In some embodiments, as discussed above, the training data 605 includes data at multiple points in time for each patient. That is, for a given patient, the training data 605 may include multiple sets of summarized clinical data 610 (one set for each relevant point in time), and the like. In some embodiments, the data contained within the summarized clinical data 610 and outcome data 620 are associated with timestamps or other indications of the relevant time or period for the data. In this way, the machine learning system 635 can identify the relevant data for any given point or window of time. For example, for a given set of summarized clinical data 610 at a given time, the machine learning system 635 can identify the relevant outcome data 620 indicating outcomes that occurred after this time.

In some embodiments, each exemplar in the training data 605 may be collectively stored in a single data structure. For example, the summarized clinical data 610 and outcome data 620 for a given time and patient may be represented or reflected as a single training exemplar, or as a sequence of data structures (e.g., a set of exemplars, each corresponding to a particular point or window in time). In some portions of the present discussion, the various components of the training data 605 are described with reference to a single patient for conceptual clarity (e.g., summarized clinical data 610 of a single patient at a single time). However, it is to be understood that the training data 605 can generally include such data for any number of patients and times.

As discussed above, the summarized clinical data 610 generally corresponds to a set of one or more salient elements of clinical data, such as specified features, attributes, or characteristics describing the patient(s). For example, the summarized clinical data 610 may include characteristics such as patient age, the biological or assigned sex of the patient, allergies the patient has, diagnoses or disorders they have, medications the patient uses, assistance they require (e.g., whether they need assistance walking), and the like. In at least one embodiment, the summarized clinical data 610 can include information or data generated by machine learning models or other techniques, as discussed above. For example, the summarized clinical data 610 may correspond to the summarized clinical data 225 of FIGS. 2-3, and may include information that was extracted from, or generated based on, natural language text (e.g., progress notes) describing the patient, as discussed above.

In the illustrated example, the outcome data 620 can generally represent or indicate data for a wide variety of outcomes with respect to each patient. For example, in some embodiments, the outcome data 620 indicates whether one or more defined (harmful) events occurred to the patient, enabling training of a machine learning model to predict the risk of such events based on summarized clinical data 610. As one example, the outcome data 620 may indicate whether the patient suffered a fall (e.g., whether they fell after returning home and/or while receiving home health services). Such data may be used to train a fall risk prediction model (e.g., to predict whether a given patient is likely to fall if they are allowed to return home and receive home health services). As another example, the outcome data 620 may indicate whether the patient was re-hospitalized (e.g., whether, while receiving home health services the patient returned to the hospital). Such data may be used to train a re-hospitalization risk prediction model (e.g., to predict whether a given patient is likely to be required to return to the hospital if they are allowed to return home and receive home health services). As yet another example, the outcome data 620 may indicate whether the patient misused or abused any medications (e.g., whether, while receiving home health services the patient began abusing prescription medications). Such data may be used to train a medication risk prediction model (e.g., to predict whether a given patient is likely to misuse their medications if they are allowed to return home and receive home health services). As yet another example, the outcome data 620 may indicate whether the patient recovered or improved (or worsened/declined) while receiving home health services. Such data may be used to train a care plan generation model (e.g., to generate care plans indicating home health services and/or to predict whether a given care plan/set of services is likely to result in improvement, stagnation, or decline in the patient's health).

Although the illustrated training data 605 includes several specific and discrete components including summarized clinical data 610 and outcome data 620, in some embodiments, the training data 605 used by the machine learning system 635 may include fewer components (e.g., a subset of the illustrated examples) or additional components not depicted.

As illustrated, the machine learning system 635 generates or trains one or more machine learning models 640 based on the training data 605. The machine learning model(s) 640 each generally specify or learn a set of parameters during training, such as weights for a neural network model. In some embodiments, the machine learning model 640 specifies weights specifically for each individual feature (e.g., for each attribute or element in the set of summarized clinical data 610). For example, a first attribute may be associated with a lower weight than a second attribute. Similarly, in some embodiments, the machine learning model 640 specifies different weights depending on the severity of the feature (e.g., depending on the severity of a disorder or diagnosis indicated in the summarized clinical data 610).

In some embodiments, the specific features considered by the machine learning model 640 (e.g., the specific elements in the summarized clinical data 610) are manually defined and curated. For example, the specific features may be defined by a subject-matter expert. In other embodiments, the specific features are learned during a training phase.

For example, the machine learning system 635 may process the training data 605 for a given patient at a given time (e.g., a set of summarized clinical data 610 at a given time for a given patient) as input to the machine learning model 640 in order to generate a predicted output (e.g., a predicted risk of one or more adverse events, a predicted outcome for a proposed set of home health services, and the like). This predicted outcome can then be compared against ground-truth data (e.g., the outcome data 620). The difference between the predicted and actual outcomes can be used to refine the parameters of the machine learning model 640, and the model can be iteratively refined (e.g., using data from multiple patients and/or multiple points in time) to accurately evaluate summarized clinical data 610 in order to predict risks and/or other outcomes.

In some embodiments, during or after training, the machine learning system 635 may prune the machine learning model 640 based in part on the learned weights. For example, if the learned weight or impact for a given feature (e.g., a specific element of the summarized clinical data 610) is below some threshold (e.g., within a threshold distance from zero), the machine learning system 635 may determine that the feature has no impact (or negligible impact) on the patient outcomes. Based on this determination, the machine learning system 635 may cull or remove this feature from the machine learning model 640 (e.g., by removing one or more neurons, in the case of a neural network). For future evaluations, the machine learning system 635 need not receive data relating to these removed features (and may refrain from processing or evaluating the data if it is received). In this way, the machine learning model 640 can be used more efficiently (e.g., with reduced computational expense and latency) to yield accurate evaluations.

In some embodiments, the machine learning system 635 may further generate an indication that the element is not probative of the predicted outcomes. In at least one embodiment, in response, the summary system may refrain from including the indicated feature(s) in future-generated summarized clinical data. That is, because the specific element does not affect the predicted outcomes (e.g., the medication risk, hospitalization risk, fall risk, and/or care plan efficacy), the summary system need not include it when generating summarized clinical data. This can improve the efficiency and reduce computational expense of the summary generation process.

In some embodiments, the machine learning system 635 can generate multiple machine learning models 640. For example, a separate machine learning model 640 may be generated for each outcome/risk (e.g., with a unique model for each specific type of risk). This may allow the machine learning system 635 to account for a variety of risk-specific changes or peculiarities. In other embodiments, the machine learning system 635 generates a universal machine learning model 640. In at least one embodiment, the machine learning model 640 may use additional considerations (e.g., location, region, and the like) as an input feature.

In some embodiments, the machine learning system 635 outputs the machine learning model 640 to one or more other systems for use. That is, the machine learning system 635 may distribute the machine learning model 640 to one or more downstream systems, where each downstream system can use the model to predict patient outcomes/risks based on their summarized clinical data. For example, the machine learning system 635 may deploy the machine learning model 640 to one or more servers associated with specific care facilities or hospitals, or associated with home health service entities, and these servers may use the model to evaluate summarized clinical data for patients of the specific facility and/or for prospective or referred patients. In at least one embodiment, the machine learning system 635 can itself use the machine learning model to evaluate clinical data across one or more locations.

### Example Workflow for Using Machine Learning Models to Evaluate Summarized Clinical Data

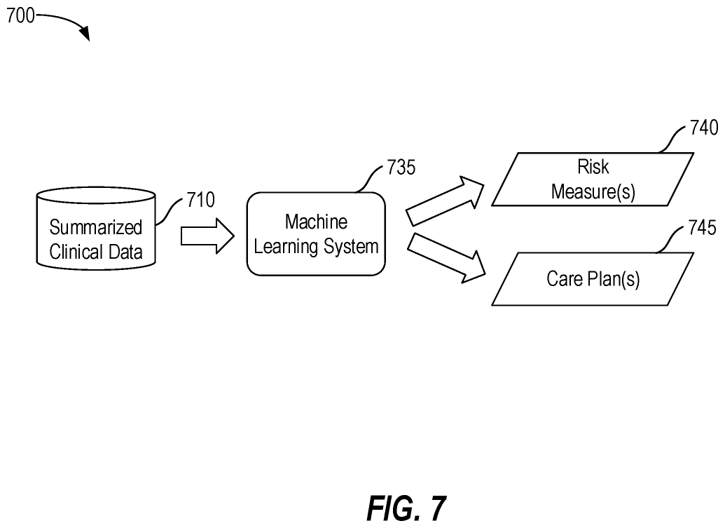

FIG. 7 depicts an example workflow 700 for using machine learning models to evaluate summarized clinical data, according to one embodiment of the present disclosure. In some embodiments, the workflow 700 is performed using trained models, such as the machine learning models 640 of FIG. 6.

In the illustrated workflow 700, a set of summarized clinical data 710 is evaluated by a machine learning system 735 using one or more machine learning models (e.g., machine learning models 640 of FIG. 6) to generate and/or evaluate one or more outcomes, such as risk measures 740 and/or care plan(s) 745. In embodiments, the machine learning system 735 may be implemented using hardware, software, or a combination of hardware and software. In some embodiments, the machine learning system 735 corresponds to the machine learning system 635 of FIG. 6. In other aspects, as discussed above, the machine learning system 735 may differ from the system that trained the model and/or generated the training data. Further, in some embodiments, the machine learning system 735 may correspond to a summary system, such as summary system 120 of FIGS. 1-2.

As discussed above, the summarized clinical data 710 generally includes information generated based on and/or extracted from patient data. In the illustrated example, the summarized clinical data 710 is processed using one or more machine learning models to generate one or more risk measures 740 and/or one or more care plans 745. Although risk measures 740 and care plans 745 are depicted for conceptual clarity, in some embodiments, the summarized clinical data 710 may be evaluated to generate a subset of the depicted predictions and/or to generate other predictions not depicted.

In the illustrated example, the risk measure(s) 740 may generally indicate the predicted risk of one or more adverse events, with respect to the patient, based on the patient's summarized clinical data 710. For example, the risk measure 740 may include a fall risk (indicating the probability that the patient will fall), a re-hospitalization risk (indicating a probability that the patient will require hospitalization), a medication risk (indicating a probability that the patient will misuse prescribed medications), and the like. In an embodiment, the predicted risks may include a categorical prediction (e.g., indicating whether it is likely, unlikely, and the like), a probability or continuous value (e.g., a score between zero and one indicating the probability), a predicted timeline (e.g., a prediction of how much time will elapse before the adverse event), and the like.

In the illustrated example, the care plan(s) 745 may generally indicate a suggested or recommended set of services (e.g., home healthcare services) generated based on the summarized clinical data 710. For example, the services may be generated or selected from a set of possible or alternatives using the trained machine learning models. In some aspects, the care plan(s) 745 may further indicate predicted outcomes, such as using categorical predictions (e.g., indicating whether the patient is predicted to improve, remain the same, or decline when receiving the services), probability predictions (e.g., a score indicating the probability of one or more outcomes), and the like.

In some embodiments, in addition to summarized clinical data 710, the machine learning system 735 may consider other data when generating risk measure(s) and/or care plan(s) 745. For example, in one embodiment, the machine learning model(s) may receive proposed care plans as input (alongside summarized clinical data 710) to generate predicted risk measures 740. As another example, in some embodiments, the machine learning model(s) may receive the summarized clinical data 710 as well as one or more predicted risks (e.g., risk measures 740 generated by other machine learning models based on summarized clinical data 710) to generate or suggest care plans 745.

In some embodiments, the machine learning system 735 can generate risk measures 740 and/or care plans according to various criteria, such as periodically (e.g., daily), when a patient referral is received, and/or when a patient transfers to a new entity. In some embodiments, the machine learning system 735 generates a new set of risk measures 740 and/or care plans 745 whenever new data becomes available (e.g., when the summarized clinical data 710 changes). For example, when the patient attribute(s) change, the machine learning system 735 may use the updated attributes to generate a new care plan 745 and/or to predict new risk measures 740. In some embodiments, whenever a patient's summarized clinical data 710 changes (e.g., due to a newly-received diagnosis), the machine learning system 735 may automatically detect the change and generate updated predictions that are specifically-tailored to the individual patient at the specific time. This targeted prophylactic treatment can significantly improve patient conditions and outcomes.

Advantageously, the automatically generated care plans 745 and risk measures 740 can significantly improve the outcomes of the patients, helping to identify potential risks or concerns and determine optimal treatment options, thereby preventing further deterioration and significantly reducing harm. Additionally, the autonomous nature of the machine learning system 735 enables improved computational efficiency and accuracy, as the risk measures 740 and/or care plans 745 are generated objectively (as opposed to the subjective judgment of clinicians or other users), as well as quickly and with minimal computational expense. That is, as the predictions can be automatically updated whenever new data is available, users need not manually retrieve and review the relevant data (which incurs wasted computational expense, as well as wasted time for the user).

Further, in some embodiments, the machine learning system 735 can regenerate care plans 745 and/or risk measures 740 during specified times (e.g., off-peak hours, such as overnight) to provide improved load balancing on the underlying computational systems. For example, rather than requiring caregivers to retrieve and review patient data repeatedly to define new plans and determine new risks, the machine learning system 735 can automatically identify such changes in the (automatically created) summarized clinical data 710, and use the machine learning model(s) to regenerate risk measures 740 and/or care plans 745 only when needed. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

In these ways, embodiments of the present disclosure can significantly improve patient outcomes while simultaneously improving the operations of the computers and/or networks themselves (at least through improved and more accurate scores and plans, as well as better load balancing of the computational burdens)

### Example Method for Generating and Evaluating Summarized Clinical Data Using Machine Learning Models

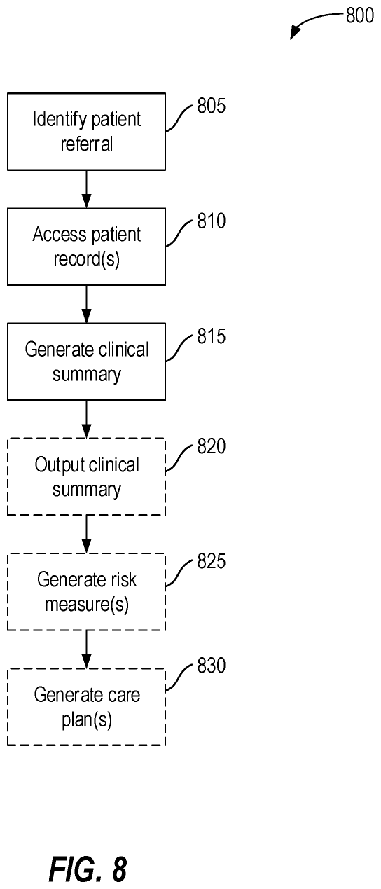

FIG. 8 is a flow diagram depicting an example method 800 for generating and evaluating summarized clinical data using machine learning models, according to one embodiment of the present disclosure. In some embodiments, the method 800 is performed by a summary system, such as the summary system 120 of FIGS. 1-2, and/or by a machine learning system, such as the machine learning system 635 of FIG. 6 and/or the machine learning system 735 of FIG. 7.

At block 805, the summary system identifies a patient referral (such as patient referral 210 of FIG. 2). For example, in some embodiments, the summary system operates as part of a healthcare facility (e.g., an entity that manages or provides home health services) and receives the patient referral from another healthcare facility (e.g., from a hospital). In some embodiments, the summary system operates as a third party system and receives the referral (or an indication of the referral) from the entity that received it (e.g., from the home healthcare facility). As discussed above, the referral may generally correspond to a request, instruction, order, suggestion, or other indication that the patient should seek and/or receive healthcare services from the indicated entity. For example, prior to or upon discharge from a hospital, the physician may provide a referral to a home health service entity to facilitate the patient receiving home health services when they return home.

At block 810, the summary system accesses one or more patient records based on the identified referral. For example, as discussed above, the summary system may request patient records for the patient identified in the referral (e.g., using a unique identifier of the referral and/or of the patient, and/or a combination of identifying information, such as the patient's name and date of birth) from the referring entity (e.g., from the hospital that provided the referral). In response, the referring entity can retrieve patient records associated with the patient and return them to the summary system.

As discussed above, the patient records can generally include a wide variety of data, including textual data, audio data, video data, and the like. For example, the patient records may include clinician or physician notes, lab reports, CCDs, faxed data, and the like.

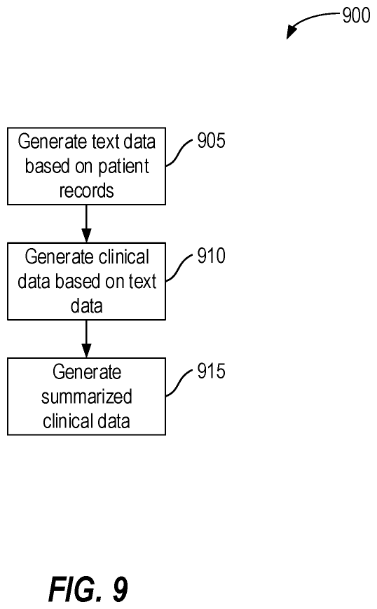

At block 815, the summary system generates a clinical summary (e.g., summarized clinical data 225 of FIGS. 1-2) for the patient based on the received records. For example, as discussed above, the summary system may use one or more ML-based models or techniques to identify, extract, and/or generate a healthcare-related summary of the patient. One example method for generating the clinical summary is discussed in more detail below with reference to FIG. 9.

At block 820, the summary system optionally outputs the clinical summary (e.g., via a GUI). For example, the healthcare provider that received the referral may request generation of the summary and review the summary displayed on the GUI for various purposes, such as to ensure that any relevant criteria are satisfied (e.g., the patient satisfies one or more defined requirements).

At block 825, the summary system optionally generates one or more risk measures, such as by processing all or a portion of the clinical summary using one or more machine learning models. For example, as discussed above, the summary system may process the clinical summary using one or more trained models to generate predicted risk measures, where the risk measure(s) indicate information such as the probability that a corresponding adverse event will occur.

At block 830, the summary system optionally generates one or more risk care plans, such as by processing all or a portion of the clinical summary using one or more machine learning models. For example, as discussed above, the summary system may process the clinical summary using one or more trained models to generate a suggested set of home healthcare services.

### Example Method for Generating Summarized Clinical Data Using Machine Learning

FIG. 9 is a flow diagram depicting an example method 900 for generating summarized clinical data using machine learning, according to one embodiment of the present disclosure. In some embodiments, the method 900 is performed by a summary system, such as the summary system 120 of FIGS. 1-2, and/or by a machine learning system, such as the machine learning system 635 of FIG. 6 and/or the machine learning system 735 of FIG. 7. In one embodiment, the method 900 provides additional detail for block 815 of FIG. 8.

At block 905, the summary system generates text data (e.g., text data 310 of FIG. 3) based on a set of patient records (e.g., patient records 220 of FIGS. 2-3). For example, as discussed above, a text ML component (such as text ML component 305 of FIG. 3) may use one or more ML models or other techniques to identify and extract or generate textual information reflected in the patient records.

At block 910, the summary system generates clinical data (e.g., clinical data 320 of FIG. 3) based on the generated text data. For example, as discussed above, an extraction ML component (such as extraction ML component 315 of FIG. 3) may use one or more ML models or other techniques to identify and extract or generate relevant or salient clinical information reflected in the text data.

At block 915, the summary system generates summarized clinical data (e.g., summarized clinical data 225 of FIGS. 2-3) based on the generated clinical data. For example, as discussed above, a summary ML component (such as summary ML component 325 of FIG. 3) may use one or more ML models or other techniques to summarize the relevant or salient clinical information reflected in the clinical data, such as by grouping associated data, removing duplicative data, and the like.

### Example Method for Generating Clinical Data Based on Extracted Text

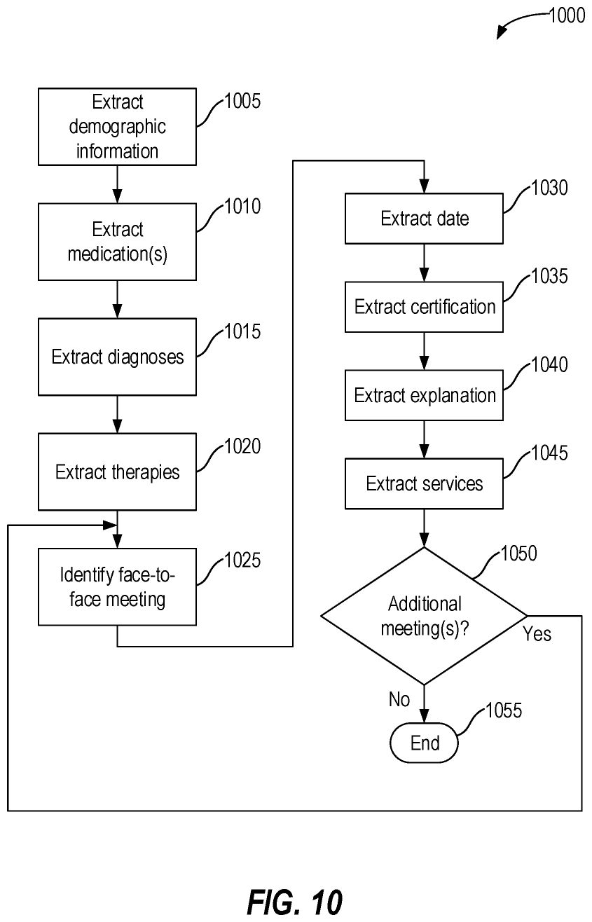

FIG. 10 is a flow diagram depicting an example method 1000 for generating clinical data based on extracted text, according to one embodiment of the present disclosure. In some embodiments, the method 1000 is performed by a summary system, such as the summary system 120 of FIGS. 1-2, and/or by a machine learning system, such as the machine learning system 635 of FIG. 6 and/or the machine learning system 735 of FIG. 7. In one embodiment, the method 1000 provides additional detail for block 910 of FIG. 9.

As discussed above, the depicted operations in FIG. 10 may be implemented using machine learning-based techniques and/or models. For example, large language models and/or prompt models may be used to identify and extract relevant information at each block 1010-1045.

At block 1005, the summary system identifies and extracts demographic information from text data generated based on and/or or extracted from the patient records. The demographic information may generally correspond to characteristics of the patient such as their age, gender, sex, race, country or region of origin, current country or region, and the like.

At block 1010, the summary system identifies and extracts medication(s) used by the patient, as reflected in the text data. As discussed above, the medication data may generally include information relating to medications that the patient currently uses and/or has used in the past, such as identifying each medication, identifying the prescribed dosages, determining how long the patient has used the medication, and the like.

At block 1015, the summary system identifies and extracts diagnoses of the patient, as reflected in the text data. As discussed above, the diagnosis data may generally include information relating to diagnoses that the patient currently has and/or has received in the past, such as identifying each diagnosis, identifying the degree or intensity of the concern, determining how long the patient has had the diagnosis, and the like.

At block 1020, the summary system identifies and extracts therapies engaged in by the patient, as reflected in the text data. As discussed above, the therapy data may generally include information relating to therapies that the patient currently engages in and/or has engaged in in the past, such as identifying each therapy, determining how long the patient has engaged in the therapy, and the like.

At block 1025, the summary system identifies one or more records (or portions of text data) relating to one or more face-to-face meetings conducted between the patient and a healthcare provider (e.g., a clinician or physician). For example, the summary system may identify defined records or documents that are used to document such face-to-face meetings.

Generally, the summary system may identify or select the face-to-face meeting using any criteria. Although depicted as a sequential process (selecting and evaluating face-to-face meetings in sequence) for conceptual clarity, in some embodiments, the summary system may select/identify/evaluate multiple such meetings in parallel.

At block 1030, the summary system identifies and extracts the date when the face-to-face meeting was conducted.

At block 1035, the summary system identifies and extracts any certifications made by the healthcare provider in conjunction with the face-to-face meeting. Such certifications may include written statements, checking or confirming defined statements (e.g., from a list of certifications), and the like. Generally, the certification(s) may indicate that the healthcare provider believes that the indicated statement is true or factual. For example, in the context of a referral for home healthcare services, the summary system may extract a certification that the healthcare provider believes that the patient should be classified as homebound, that the patient reasonably needs home healthcare services, and the like.

At block 1040, the summary system identifies and extracts any explanations provided by the healthcare provider in conjunction with the meeting and/or with the certifications. Such explanations may include, for example, written or typed natural language text. For example, the healthcare provider may indicate the reasoning as to why the patient is homebound (e.g., because they are bedridden, or otherwise have substantial difficulty moving).

At block 1045, the summary system identifies and extracts any suggested or indicated services recommended by the healthcare provider in conjunction with the meeting. Such explanations may include, for example, written or typed natural language text, checking or selection from a defined list, and the like. For example, the healthcare provider may indicate the that the patient should receive (or may benefit from) services such as movement assistance, cleaning and/or cooking assistance, medication assistance, and the like.

In some aspects, the summary system may extract a wide variety of information for the face-to-face meeting, including data not depicted in the illustrated example. For example, the summary system may determine how much time has elapsed since the meeting, may identify the physician that conducted/participated in the meeting, and the like.

At block 1050, the summary system determines whether there are any other face-to-face meetings reflected in the textual data. If so, the method 1000 returns to block 1025 to select another meeting for evaluation. If not, the method 1000 terminates at block 1055.

### Example Method for Generating Summarized Clinical Data

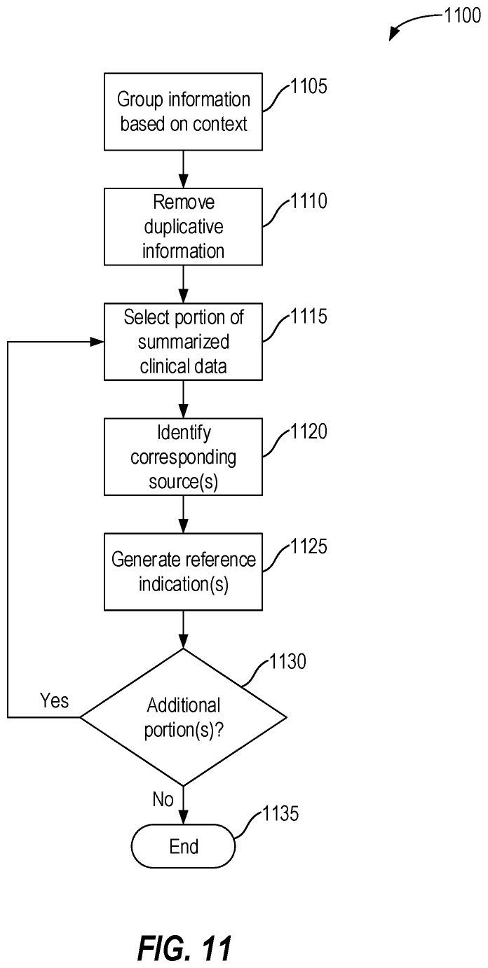

FIG. 11 is a flow diagram depicting an example method 1100 for generating summarized clinical data, according to one embodiment of the present disclosure. In some embodiments, the method 1100 is performed by a summary system, such as the summary system 120 of FIGS. 1-2, and/or by a machine learning system, such as the machine learning system 635 of FIG. 6 and/or the machine learning system 735 of FIG. 7. In one embodiment, the method 1100 provides additional detail for block 915 of FIG. 9.

As discussed above, the depicted operations in FIG. 11 may be implemented using machine learning-based techniques and/or models. For example, large language models and/or prompt models may be used to identify, extract, group, and/or de-duplicate relevant concepts and clinical information at each block 1105-1125.

At block 1105, the summary system groups information reflected in the clinical data based on contextual information. In some embodiments, as discussed above, the summary system may use one or more machine learning models to cluster the various elements or portions of the clinical data to identify groups or clusters of data that are contextually similar and/or should be presented or summarized together. For example, the summary system may group diagnoses (which may be reflected in a wide variety of concepts or text data) together under a single group. As another example, the summary system may group data related to a single meeting or other event together. In some embodiments, a single element of clinical data may be associated with or assigned to multiple groups. Generally, the specific groupings used may vary depending on the particular implementation.

At block 1110, the summary system removes duplicative information from the grouped information. For example, if a single diagnosis is represented or reflected multiple times (e.g., where multiple patient records refer to the same diagnosis), the summary system may de-duplicate this data by removing or deleting one or more of the instances from the summarized data. That is, the summary system can ensure that the summarized data only includes or indicates the diagnosis once, as opposed to repeating it multiple times.

At block 1115, the summary system selects a portion or element of the summarized clinical data. As discussed above, each element in the summarized data generally corresponds to a single logical piece of data, such as a characteristic or attribute of the patient. For example, one element may be a first specific diagnosis, while a second element is another diagnosis and a third element is the patient's age.

Generally, the summary system may select the element of the summarized data using any suitable criteria, including randomly or pseudo-randomly, as all elements will be processed during the method 1100. Although depicted as a sequential process (selecting and evaluating elements in sequence) for conceptual clarity, in some embodiments, the summary system may select/identify/evaluate multiple such elements in parallel.

At block 1120, the summary system identifies one or more corresponding sources for the selected element of summarized clinical data. In some embodiments, as discussed above, the source patient record(s) of each element may be generated during prior operations and maintained during processing. For example, when text data is generated/extracted, the summary system may generate metadata indicating the record(s) from which the text was generated/extracted. Similarly, when clinical concepts are generated or extracted, the summary system may generate or maintain this metadata to indicate the original source record(s). Subsequently, when summarizing the data, the summary system may generate or use this metadata at block 1120.

In some embodiments, identifying the source(s) can include identifying the most-recent patient document that reflects, supports, or otherwise indicates that the element is a proper or accurate part of the summarized clinical information. In some embodiments, identifying the source(s) can include identifying all patient documents that support the element. Additionally, in some embodiments, identifying the source(s) includes identifying the overall document/record. In some embodiments, the summary system may further identify specific portion(s) of documents or records, such as by page number, section number, and the like.

At block 1125, the summary system generates one or more reference indications for the element, indicating the identified source(s). As discussed above, the format and content of the reference indications may vary depending on the particular implementation. Generally, the reference indications are used to indicate the portions of patient data (e.g., records or documents) that support, disclose, include, or otherwise indicate the element. In some embodiments, the reference indicators are included as metadata with the summarized clinical data. In some embodiments, the reference indicators are included using footnotes and/or endnotes associated with each element. In some embodiments, the reference indicators are included directly in the text of the summarized data.

In some embodiments, each reference indication identifies the supporting documentation using unique identifiers, document names/dates, and the like. In some embodiments, the reference indications can include pointers or links to the source documentation, allowing for rapid access and review.

In this way, for any given element in the summarized data, the user may be able to readily identify the supporting documentation (e.g., by clicking the indication, hovering their mouse or other input device over the element, and the like).

### Example Method for Preprocessing Unstructured Input Data to Improve Machine Learning Results

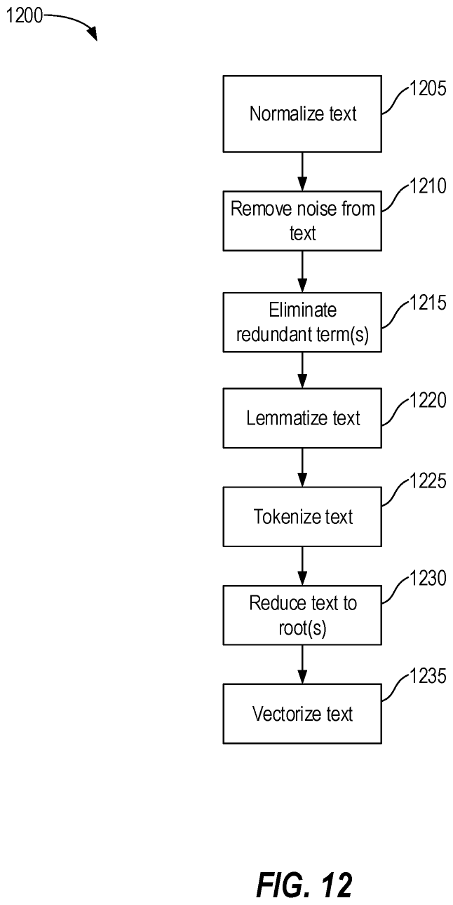

FIG. 12 is a flow diagram depicting an example method 1200 for preprocessing unstructured input data to improve machine learning results, according to one embodiment of the present disclosure. In some embodiments, the method 1200 is performed by a summary system, such as the summary system 120 of FIGS. 1-2, and/or by a machine learning system, such as the machine learning system 635 of FIG. 6 and/or the machine learning system 735 of FIG. 7.

Generally, each block in the method 1200 is optional, and the summary system may perform all of the indicated operations, or some subset thereof. The summary system may also use additional preprocessing steps not depicted in the illustrated example. Additionally, though the illustrated example suggests a linear and sequential process for conceptual clarity, in embodiments, the operations may be performed in any order (including entirely or partially in parallel). Additionally, though described as “preprocessing,” in some aspects, some or all of the depicted operations may be performed as part of data processing by the summary system to generate clinical summaries.

In an embodiment, the method 1200 can be used to preprocess natural language text extracted from written notes, such as progress notes authored by a physician. For example, a healthcare provider may write notes relating to a patient's progress, such as whether a disease is progressing, improving, or stagnant, whether any adverse events or concerns (such as nausea) were reported by the patient, and the like. This extracted text can be processed to generate summarized clinical data that enables efficient gleaning of insights useful for a variety of purposes, as discussed above, but may require some level of preprocessing first in some aspects.

At block 1205, the summary system can normalize the extracted natural language text. As discussed above, this normalization may include a wide variety of text normalization processes, such as converting all characters in the extracted text to lowercase, converting accented or foreign language characters to ASCII characters, expanding contractions, converting words to numeric form where applicable, converting dates to a standard date format, and the like.

At block 1210, the summary system removes noise from the text. As discussed above, noise removal may include identification and removal of portions of the extracted text that do not carry meaningful or probative value, such as characters, portions, or elements of the text that are not useful or meaningful in the ultimate computing task (e.g., predicting medication efficacy), and/or that are not useful to human readers. For example, the noise removal may include removing extra white or blank spaces, tabs, or lines, removing tags such as HTML tags, and the like.

At block 1215, the summary system can eliminate redundant elements or terms from the text. As discussed above, this may include identifying and eliminating or removing text corresponding to redundant elements (e.g., duplicate words), and/or the reduction of a sentence or phrase to a portion thereof that is most suitable for machine learning training or application. For example, the redundancy elimination may include eliminating verbs (which may be unhelpful in the desired tasks), conjunctions, or other extraneous words that do not aid the task.

At block 1220, the summary system lemmatizes the text. As discussed above, text lemmatization can generally include stemming and/or lemmatization of one or more words in the extracted text. This may include converting words from their inflectional or other form to a base form. For example, lemmatization may include replacing “holding,” “holds,” and “held” with the base form “hold.”

At block 1225, the summary system tokenizes the text. In an embodiment, tokenizing the text may include transforming or splitting elements in the extracted text (e.g., strings of characters) into smaller elements, also referred to as “tokens.” For example, the tokenization may include tokenizing a paragraph into a set of sentences, tokenizing a sentence into a set of words, transforming a word into a set of characters, and the like. In some embodiments, tokenization can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

At block 1230, the summary system can reduce the text to one or more roots. As discussed above, the root generation can include reducing portion of the extracted text (e.g., a phrase or sentence) to its most relevant n-gram (e.g., a bigram) or root for downstream machine learning training and/or application.

At block 1235, the summary system can vectorize the text. Generally, vectorization may include converting the text into one or more objects that can be represented numerically (e.g., into a vector or tensor form). For example, the summary system may use one-hot encodings (e.g., where each element in the vector indicates the presence or absence of a given keyword, phrase, sentiment, or other concept, based on the value of the element). In some embodiments, the summary system can generate one or more word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text.

### Example Method for Training Machine Learning Models to Evaluate Summarized Clinical Data

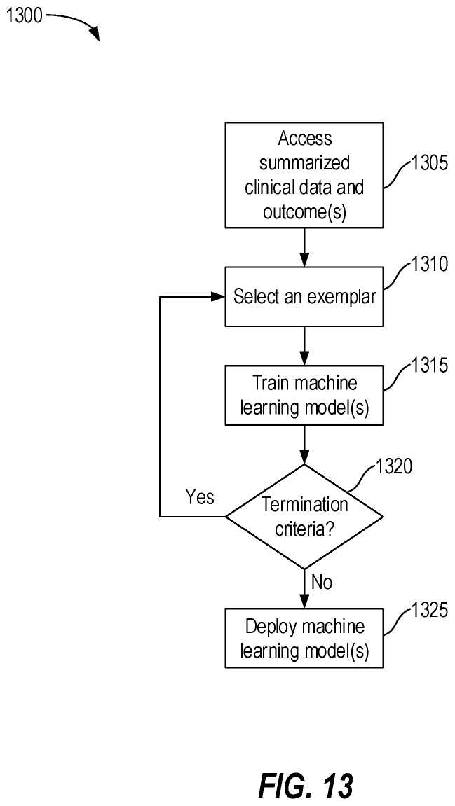

FIG. 13 is a flow diagram depicting an example method 1300 for training machine learning models to evaluate summarized clinical data, according to one embodiment of the present disclosure. In some embodiments, the method 1300 is performed by a summary system, such as the summary system 120 of FIGS. 1-2, and/or by a machine learning system, such as the machine learning system 635 of FIG. 6 and/or the machine learning system 735 of FIG. 7. In one embodiment, the method 1300 can be used to generate or train machine learning models used during blocks 825 and/or 830 of FIG. 8.

At block 1305, the machine learning system accesses summarized clinical data (e.g., summarized clinical data 610 of FIG. 6) and outcome data (e.g., outcome data 615 of FIG. 6). For example, in some embodiments, the machine learning system generates the summarized clinical data. In some embodiments, the machine learning system accesses it from another system or repository. As discussed above, the machine learning system may generally access training records or exemplars for training or refining one or more machine learning models that process summarized clinical data as input.

At block 1310, the machine learning system selects one of the exemplars. As discussed above, each exemplar may generally include a set of summarized clinical data for a patient (e.g., generated as discussed above), as well as information relating to outcome(s) experienced by the patient after the clinical data was summarized, such as whether they suffered an adverse event (e.g., a fall), whether they recovered or declined in health, and the like. Generally, the machine learning system may select the exemplar using any suitable criteria, including randomly or pseudo-randomly, as all exemplars swill be processed during the method 1300. Although depicted as a sequential process (selecting and evaluating exemplars in sequence) for conceptual clarity, in some embodiments, the machine learning system may select/identify/evaluate multiple such exemplars in parallel.

At block 1315, the machine learning system trains one or more machine learning models based on the selected exemplar(s). Generally, the particular operations and techniques used to train the machine learning model(s) may vary depending on the particular implementation. For example, in some embodiments, the machine learning system processes the summarized clinical data from the exemplar as input using the machine learning model to generate an output (e.g., a predicted outcome), and compares the predicted outcome with a ground-truth outcome reflected in the exemplar. Based on the difference, the machine learning system may refine the parameters of the model. For example, in the case of a neural network architecture, the machine learning system may compute a loss based on the difference, and use backpropagation to refine the weights and/or biases of the network. Although the illustrated example depicts sequentially training the model(s) based on each exemplar separately (e.g., using stochastic gradient descent), in some aspects, the machine learning system may use multiple exemplars to refine the model simultaneously (e.g., using batch gradient descent).

At block 1320, the machine learning system determines whether one or more termination criteria are met. Generally, determining whether the termination criteria are satisfied may vary depending on the particular implementation, and may include considerations such as determining whether additional exemplar(s) remain, determining whether a desired model accuracy has been reached, determining whether a defined amount of time or computational resources have been spent training, and the like. If the criteria are not satisfied, the method 1300 returns to block 1310. If the criteria are satisfied, the method 1300 continues to block 1325.

At block 1325, the machine learning system deploys the machine learning model(s) for inferencing. Generally, deploying the model(s) can include a wide variety of operations, such as transmitting the trained model(s) (e.g., transmitting the parameters of the model) to one or more inferencing systems, instantiating the model for local inferencing, and the like.

In some embodiments, as discussed above, the machine learning system may train one or more machine learning models to generate or predict one or more outcomes. For example, one model may be trained to predict one or more risk measures, while a second model is trained to generate care plans. In some embodiments, separate models (or model components) may be trained for each type of risk (e.g., one model for fall risk, one for hospitalization risk, and the like).

### Example Method for Using Machine Learning Models to Evaluate Summarized Clinical Data

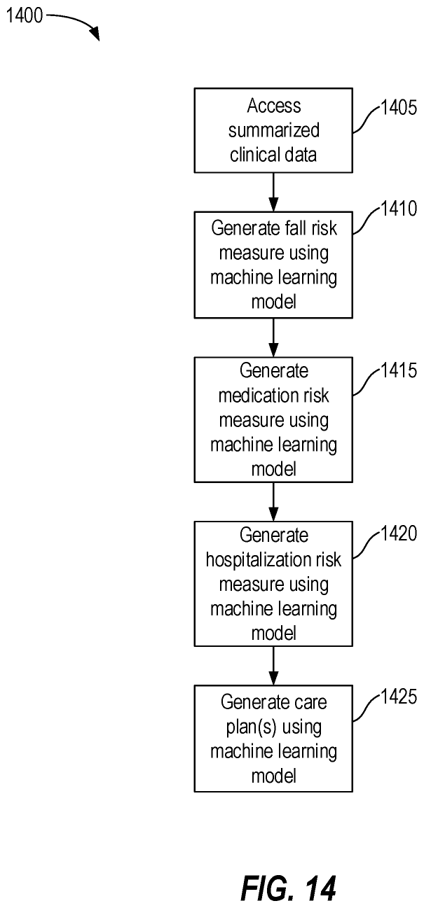

FIG. 14 is a flow diagram depicting an example method 1400 for using machine learning models to evaluate summarized clinical data, according to one embodiment of the present disclosure. In some embodiments, the method 1400 is performed by a summary system, such as the summary system 120 of FIGS. 1-2, and/or by a machine learning system, such as the machine learning system 635 of FIG. 6 and/or the machine learning system 735 of FIG. 7. In one embodiment, the method 1400 provides additional detail for blocks 825 and/or 830 of FIG. 8.

At block 1405, the machine learning system accesses summarized clinical data for a patient. For example, the machine learning system may generate the summarized clinical data, or may receive it from another system or component.

At block 1410, the machine learning system generates a fall risk measure using a trained machine learning model (e.g., using a fall risk prediction machine learning model). For example, the machine learning system may input the summarized clinical data to the risk prediction model to generate a fall risk measure indicating whether the patient is at risk of a fall, a degree or probability of the risk, and the like.

At block 1415, the machine learning system generates a medication risk measure using a trained machine learning model (e.g., using a medication risk prediction machine learning model). For example, the machine learning system may input the summarized clinical data to the risk prediction model to generate a medication risk measure indicating whether the patient is likely to misuse prescribed medications, a degree or probability of the risk, and the like.

At block 1420, the machine learning system generates a hospitalization risk measure using a trained machine learning model (e.g., using a hospitalization risk prediction machine learning model). For example, the machine learning system may input the summarized clinical data to the risk prediction model to generate a hospitalization risk measure indicating whether the patient is at risk of re-hospitalization, a degree or probability of the risk, and the like.

At block 1425, the machine learning system generates a care plan using a trained machine learning model (e.g., using a care plan generation machine learning model). For example, the machine learning system may input the summarized clinical data to the care plan model to generate a set of home health services that would benefit the patient. In some embodiments, the machine learning system may additionally or alternatively provide other data as input to the care plan model. For example, in one embodiment, the machine learning system may input the summarized clinical data as well as one or more risk measures (e.g., the fall risk measure generated at block 1410, the medication risk measure generated at block 1415, and/or the hospitalization risk measure generated at block 1420).

### Example Method for Generating and Outputting Summarized Clinical Data

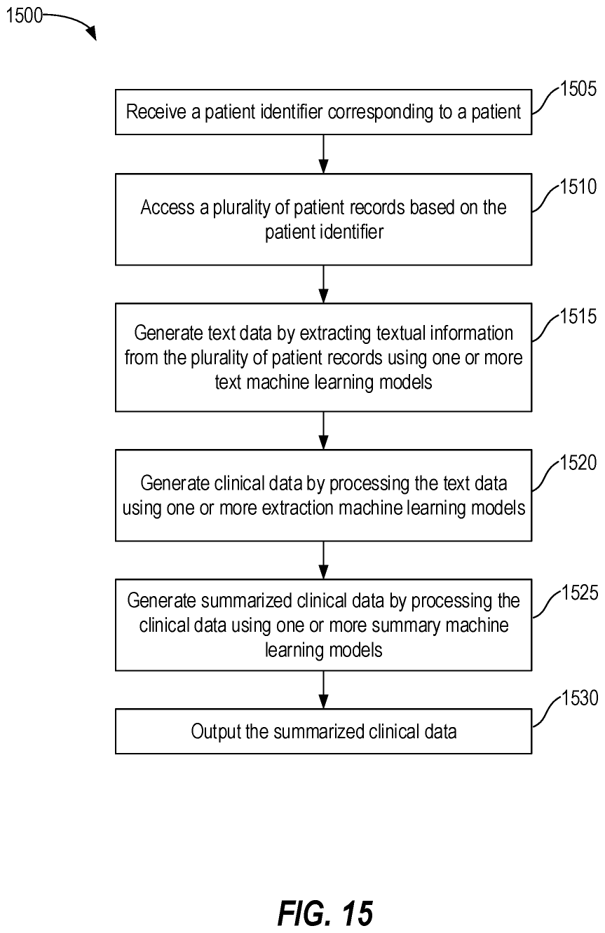

FIG. 15 is a flow diagram depicting an example method 1500 for generating and outputting summarized clinical data, according to one embodiment of the present disclosure. In some embodiments, the method 1500 is performed by a summary system, such as the summary system 120 of FIGS. 1-2, and/or by a machine learning system, such as the machine learning system 635 of FIG. 6 and/or the machine learning system 735 of FIG. 7.

At block 1505, a patient identifier corresponding to a patient is received (e.g., from a patient referral, such as patient referral 210 of FIG. 2).

At block 1510, a plurality of patient records (e.g., patient records 220 of FIG. 2) is accessed based on the patient identifier.

At block 1515, text data (e.g., text data 310 of FIG. 3) is generated by extracting textual information from the plurality of patient records using one or more text machine learning models (e.g., text ML component 305 of FIG. 3).

At block 1520, clinical data (e.g., clinical data 320 of FIG. 3) is generated by processing the text data using one or more extraction machine learning models (e.g., extraction ML component 315 of FIG. 3).

At block 1525, summarized clinical data (e.g., summarized clinical data 225 of FIG. 3) is generated by processing the clinical data using one or more summary machine learning models (e.g., summary ML component 325 of FIG. 3).

At block 1530, the summarized clinical data is output.

### Example Method for Training Machine Learning Models Based on Summarized Clinical Data

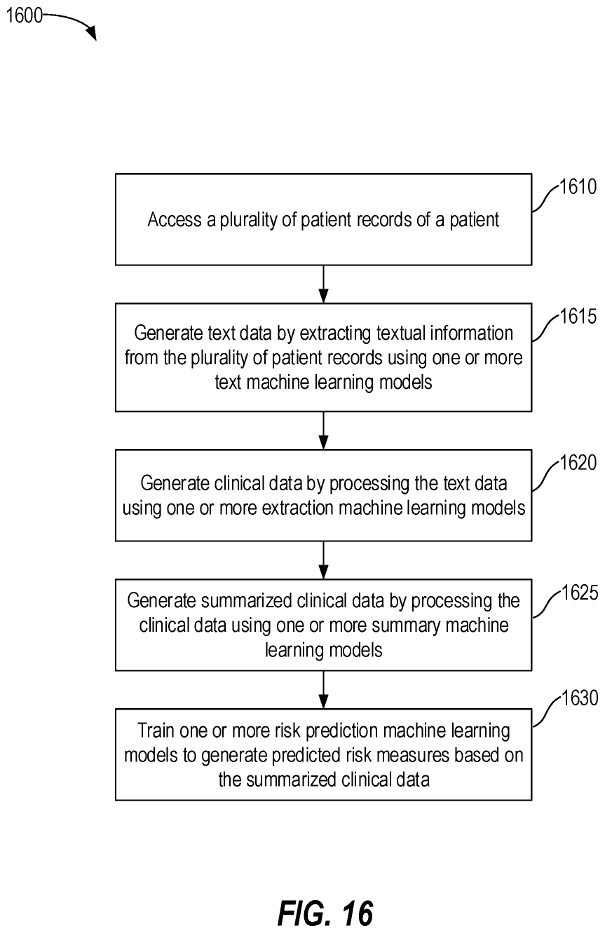

FIG. 16 is a flow diagram depicting an example method 1600 for training machine learning models based on summarized clinical data, according to one embodiment of the present disclosure. In some embodiments, the method 1600 is performed by a summary system, such as the summary system 120 of FIGS. 1-2, and/or by a machine learning system, such as the machine learning system 635 of FIG. 6 and/or the machine learning system 735 of FIG. 7.

At block 1610, a plurality of patient records (e.g., patient records 220 of FIG. 2) is accessed based on the patient identifier.

At block 1615, text data (e.g., text data 310 of FIG. 3) is generated by extracting textual information from the plurality of patient records using one or more text machine learning models (e.g., text ML component 305 of FIG. 3).

At block 1620, clinical data (e.g., clinical data 320 of FIG. 3) is generated by processing the text data using one or more extraction machine learning models (e.g., extraction ML component 315 of FIG. 3).

At block 1625, summarized clinical data (e.g., summarized clinical data 225 of FIG. 3) is generated by processing the clinical data using one or more summary machine learning models (e.g., summary ML component 325 of FIG. 3).

At block 1630, one or more risk prediction machine learning models (e.g., machine learning models 640 of FIG. 6) are trained to generate predicted risk measures (e.g., risk measures 740 of FIG. 7) based on the summarized clinical data.

### Example Computing Device for Generating and Evaluating Summarized Clinical Data Using Machine Learning

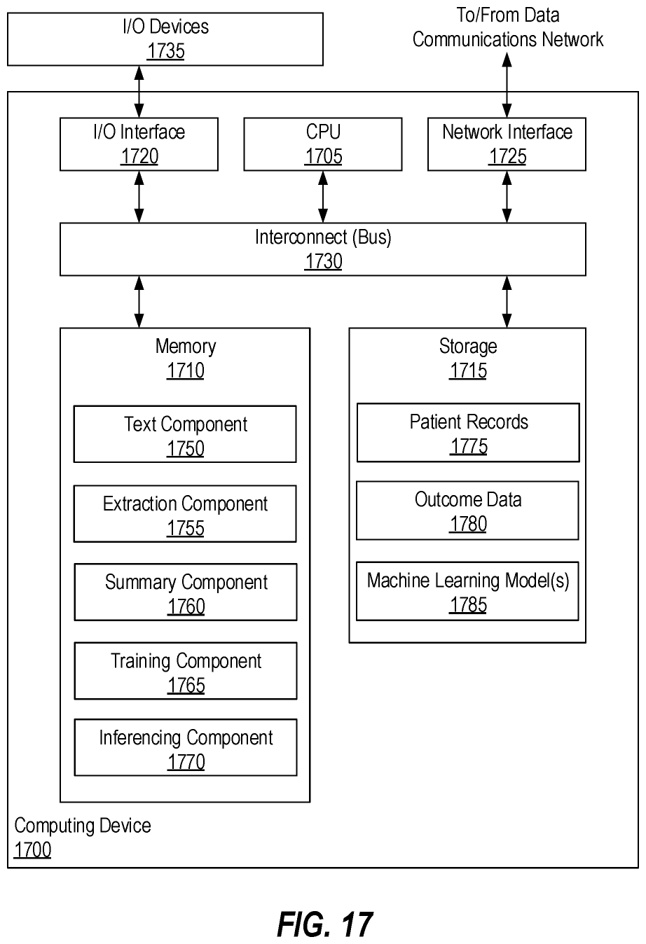

FIG. 17 depicts an example computing device 1700 configured to perform various aspects of the present disclosure. Although depicted as a physical device, in embodiments, the computing device 1700 may be implemented using virtual device(s), and/or across a number of devices (e.g., in a cloud environment). In one embodiment, the computing device 1700 corresponds to or implements the summary system 120 of FIGS. 1-2, the machine learning system 635 of FIG. 6, and/or the machine learning system 735 of FIG. 7.

As illustrated, the computing device 1700 includes a CPU 1705, memory 1710, a network interface 1725, and one or more I/O interfaces 1720. Though not included in the depicted example, in some embodiments, the computing device 1700 also includes one or more storages. In the illustrated embodiment, the CPU 1705 retrieves and executes programming instructions stored in memory 1710, as well as stores and retrieves application data residing in memory 1710 and/or storage (not depicted). The CPU 1705 is generally representative of a single CPU and/or GPU, multiple CPUs and/or GPUs, a single CPU and/or GPU having multiple processing cores, and the like. The memory 1710 is generally included to be representative of a random access memory. In an embodiment, if storage is present, it may include any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1735 (such as keyboards, monitors, etc.) are connected via the I/O interface(s) 1720. Further, via the network interface 1725, the computing device 1700 can be communicatively coupled with one or more other devices and components (e.g., via a network, which may include the Internet, local network(s), and the like). As illustrated, the CPU 1705, memory 1710, network interface(s) 1725, and I/O interface(s) 1720 are communicatively coupled by one or more buses 1730.

In the illustrated embodiment, the memory 1710 includes a text component 1750, an extraction component 1755, a summary component 1760, a training component 1765, and an inferencing component 1770, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1710, in embodiments, the operations of the depicted components (and others not illustrated) may be implemented using hardware, software, or a combination of hardware and software.

For example, the text component 1750 (which may correspond to the text ML component 305 of FIG. 3) may generally be used to identify, extract, and/or generate textual data (e.g., text data 310 of FIG. 3) from patient records, as discussed above. The extraction component 1755 (which may correspond to the extraction ML component 315 of FIG. 3) may generally be used to identify, extract, and/or generate clinical data (e.g., clinical data 320 of FIG. 3) from text data, as discussed above. The summary component 1760 (which may correspond to the summary ML component 325 of FIG. 3) may generally be used to summarize clinical data (e.g., to generate summarized clinical data 225 of FIG. 3) from clinical data, as discussed above.

The training component 1765 (which may correspond to the machine learning system 635 of FIG. 6) may be used to train one or more machine learning models (such as machine learning models 1785) based on summarized clinical data, as discussed above. The inferencing component 1770 (which may correspond to the machine learning system 735 of FIG. 7) may be used to generate outcome predictions by processing summarized clinical data using one or more machine learning models (such as machine learning models 1785), as discussed above.

In the illustrated example, the storage 1715 includes patient records 1775, outcome data 1780, and machine learning models 1785. Although depicted as residing in storage 1714, the depicted data may be stored in any suitable location. In at least one embodiment, as discussed above, the patient records 1775, outcome data 1780, and machine learning models 1785 may be stored in separate repositories.

Generally, the patient records 1775 may include healthcare-related information for one or more patients, as discussed above. This may include patient records for current patients (e.g., patient records 110 of FIG. 1) and/or training data from prior times (e.g., training data 605 of FIG. 6). Although not included in the illustrated example, in some embodiments, the storage 1715 may further include summarized clinical data generated based on one or more of the patient records 1775. The outcome data 1780 (which may correspond to outcome data 620 of FIG. 6) may include information relating to outcomes experienced by patients, such as whether they suffered an adverse event, whether they improved using home health services, and the like. The machine learning model(s) 1785 may generally correspond to one or more models trained to generate output based on input summarized clinical data, such as one or more risk prediction models, one or more care plan generation or suggestions models, and the like.

### Example Clauses

Clause 1: A method, comprising: receiving a patient identifier corresponding to a patient; accessing a plurality of patient records based on the patient identifier; generating text data by extracting textual information from the plurality of patient records using one or more text machine learning models; generating clinical data by processing the text data using one or more extraction machine learning models; generating summarized clinical data by processing the clinical data using one or more summary machine learning models; and outputting the summarized clinical data.

Clause 2: The method of Clause 1, wherein receiving the patient identifier comprises receiving a patient referral, for the patient, for home health services.

Clause 3: The method of any one of Clauses 1-2, wherein accessing the plurality of patient records comprises transmitting, to a referring entity that provided the patient referral, one or more requests for the plurality of patient records in response to receiving the patient referral.

Clause 4: The method of any one of Clauses 1-3, wherein the plurality of patient records comprise at least one of: (i) clinician notes, (ii) faxed documents, (iii) a continuity of care document (CCD), or (iv) one or more lab reports.

Clause 5: The method of any one of Clauses 1-4, wherein generating the clinical data comprises extracting, from the text data, at least one of: (i) demographic information of the patient, (ii) diagnoses of the patient, (iii) medications used by the patient, or (iv) therapies that the patient engages in.

Clause 6: The method of any one of Clauses 1-5, wherein generating the clinical data comprises extracting information corresponding to a face-to-face meeting between the patient and a clinician, the information comprising at least one of: (i) a date when the face-to-face meeting was conducted, (ii) a certification, by the clinician, indicating that the patient would benefit from receiving home health services, (iii) an explanation of why the patient would benefit from home health services, or (iv) an indication of one or more recommended home health services.

Clause 7: The method of any one of Clauses 1-6, wherein generating the summarized clinical data comprises at least one of: (i) grouping information, from the clinical data, based on contextual information, or (ii) removing duplicative information from the grouped information.

Clause 8: The method of any one of Clauses 1-7, wherein outputting the summarized clinical data comprises: generating a summary document comprising the summarized clinical data; embedding one or more reference indications in the summary document, wherein each respective reference indication corresponds to a respective element of the summarized clinical data and identifies one or more source records, from the plurality of patient records, from which the clinical data was extracted; and outputting summary document via a graphical user interface (GUI).

Clause 9: The method of any one of Clauses 1-8, wherein outputting the summarized clinical data comprises generating one or more predicted risk measures based on processing the summarized clinical data using one or more risk prediction machine learning models.

Clause 10: The method of any one of Clauses 1-9, wherein the one or more predicted risk measures comprise at least one of: (i) a medication risk, (ii) a re-hospitalization risk, or (iii) a fall risk.

Clause 11: The method of any one of Clauses 1-10, wherein outputting the summarized clinical data comprises generating a recommended care plan based on processing the summarized clinical data using one or more care plan generation machine learning models.

Clause 12: The method of any one of Clauses 1-11, wherein: the recommended care plan is generated based further on one or more predicted risk measures generated using one or more risk prediction machine learning models, and the predicted risk measures comprise at least one of: (i) a medication risk, (ii) a re-hospitalization risk, or (iii) a fall risk.

Clause 13: A method, comprising: accessing a plurality of patient records of a patient; generating text data by extracting textual information from the plurality of patient records using one or more text machine learning models; generating clinical data by processing the text data using one or more extraction machine learning models; generating summarized clinical data by processing the clinical data using one or more summary machine learning models; and training one or more risk prediction machine learning models to generate predicted risk measures based on the summarized clinical data.

Clause 14: The method of Clause 13, wherein accessing the plurality of patient records comprises transmitting, to a referring entity that provided a patient referral to a home health services entity, one or more requests for the plurality of patient records.

Clause 15: The method of any one of Clauses 13-14, wherein the plurality of patient records comprise at least one of: (i) clinician notes, (ii) faxed documents, (iii) a continuity of care document (CCD), or (iv) one or more lab reports.

Clause 16: The method of any one of Clauses 13-15, wherein generating the clinical data comprises extracting, from the text data, at least one of: (i) demographic information of the patient, (ii) diagnoses of the patient, (iii) medications used by the patient, or (iv) therapies that the patient engages in.

Clause 17: The method of any one of Clauses 13-16, wherein generating the clinical data comprises extracting information corresponding to a face-to-face meeting between the patient and a clinician, the information comprising at least one of: (i) a date when the face-to-face meeting was conducted, (ii) a certification, by the clinician, indicating that the patient would benefit from receiving home health services, (iii) an explanation of why the patient would benefit from home health services, or (iv) an indication of one or more recommended home health services.

Clause 18: The method of any one of Clauses 13-17, wherein generating the summarized clinical data comprises at least one of: (i) grouping information, from the clinical data, based on contextual information, or (ii) removing duplicative information from the grouped information.

Clause 19: The method of any one of Clauses 13-18, wherein the predicted risk measures comprise at least one of: (i) a medication risk, (ii) a re-hospitalization risk, or (iii) a fall risk.

Clause 20: The method of any one of Clauses 13-19, the method further comprising training one or more care plan generation machine learning models to generate recommended care plans based on the summarized clinical data and predicted risk measures.

Clause 21: A system, comprising: a memory comprising computer-executable instructions; and one or more processors configured to execute the computer-executable instructions and cause the processing system to perform an operation in accordance with any one of Clauses 1-20.

Clause 22: A system, comprising means for performing a method in accordance with any one of Clauses 1-20.

Clause 23: A non-transitory computer-readable medium comprising computer-executable instructions that, when executed by one or more processors of a processing system, cause the processing system to perform a method in accordance with any one of Clauses 1-20.

Clause 24: A computer program product embodied on a computer-readable storage medium comprising code for performing a method in accordance with any one of Clauses 1-20.

### ADDITIONAL CONSIDERATIONS

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., the components of the summary system 120 of FIGS. 1-2) or related data available in the cloud. For example, the summary system 120 could execute on a computing system in the cloud and generate summarized clinical data. In such a case, the summary system 120 could store the summarized data and/or other data at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
receiving a patient identifier corresponding to a patient;
accessing a plurality of patient records based on the patient identifier;
generating text data by extracting textual information from the plurality of patient records using one or more text machine learning models;
generating clinical data by processing the text data using one or more extraction machine learning models;
generating summarized clinical data by processing the clinical data using one or more summary machine learning models; and
outputting the summarized clinical data.

2. The method of claim 1, wherein receiving the patient identifier comprises receiving a patient referral, for the patient, for home health services.

3. The method of claim 2, wherein accessing the plurality of patient records comprises transmitting, to a referring entity that provided the patient referral, one or more requests for the plurality of patient records in response to receiving the patient referral.

4. The method of claim 1, wherein the plurality of patient records comprise at least one of: (i) clinician notes, (ii) faxed documents, (iii) a continuity of care document (CCD), or (iv) one or more lab reports.

5. The method of claim 1, wherein generating the clinical data comprises extracting, from the text data, at least one of: (i) demographic information of the patient, (ii) diagnoses of the patient, (iii) medications used by the patient, or (iv) therapies that the patient engages in.

6. The method of claim 1, wherein generating the clinical data comprises extracting information corresponding to a face-to-face meeting between the patient and a clinician, the information comprising at least one of: (i) a date when the face-to-face meeting was conducted, (ii) a certification, by the clinician, indicating that the patient would benefit from receiving home health services, (iii) an explanation of why the patient would benefit from home health services, or (iv) an indication of one or more recommended home health services.

7. The method of claim 1, wherein generating the summarized clinical data comprises at least one of: (i) grouping information, from the clinical data, based on contextual information, or (ii) removing duplicative information from the grouped information.

8. The method of claim 1, wherein outputting the summarized clinical data comprises:
generating a summary document comprising the summarized clinical data;
embedding one or more reference indications in the summary document, wherein each respective reference indication corresponds to a respective element of the summarized clinical data and identifies one or more source records, from the plurality of patient records, from which the clinical data was extracted; and
outputting summary document via a graphical user interface (GUI).

9. The method of claim 1, wherein outputting the summarized clinical data comprises generating one or more predicted risk measures based on processing the summarized clinical data using one or more risk prediction machine learning models.

10. The method of claim 9, wherein the one or more predicted risk measures comprise at least one of: (i) a medication risk, (ii) a re-hospitalization risk, or (iii) a fall risk.

11. The method of claim 1, wherein outputting the summarized clinical data comprises generating a recommended care plan based on processing the summarized clinical data using one or more care plan generation machine learning models.

12. The method of claim 11, wherein:
the recommended care plan is generated based further on one or more predicted risk measures generated using one or more risk prediction machine learning models, and
the predicted risk measures comprise at least one of: (i) a medication risk, (ii) a re-hospitalization risk, or (iii) a fall risk.

13. A method, comprising:
accessing a plurality of patient records of a patient;
generating text data by extracting textual information from the plurality of patient records using one or more text machine learning models;
generating clinical data by processing the text data using one or more extraction machine learning models;
generating summarized clinical data by processing the clinical data using one or more summary machine learning models; and
training one or more risk prediction machine learning models to generate predicted risk measures based on the summarized clinical data.

14. The method of claim 13, wherein accessing the plurality of patient records comprises transmitting, to a referring entity that provided a patient referral to a home health services entity, one or more requests for the plurality of patient records.

15. The method of claim 13, wherein the plurality of patient records comprise at least one of: (i) clinician notes, (ii) faxed documents, (iii) a continuity of care document (CCD), or (iv) one or more lab reports.

16. The method of claim 13, wherein generating the clinical data comprises extracting, from the text data, at least one of: (i) demographic information of the patient, (ii) diagnoses of the patient, (iii) medications used by the patient, or (iv) therapies that the patient engages in.

17. The method of claim 13, wherein generating the clinical data comprises extracting information corresponding to a face-to-face meeting between the patient and a clinician, the information comprising at least one of: (i) a date when the face-to-face meeting was conducted, (ii) a certification, by the clinician, indicating that the patient would benefit from receiving home health services, (iii) an explanation of why the patient would benefit from home health services, or (iv) an indication of one or more recommended home health services.

18. The method of claim 13, wherein generating the summarized clinical data comprises at least one of: (i) grouping information, from the clinical data, based on contextual information, or (ii) removing duplicative information from the grouped information.

19. The method of claim 13, wherein the predicted risk measures comprise at least one of: (i) a medication risk, (ii) a re-hospitalization risk, or (iii) a fall risk.

20. The method of claim 13, the method further comprising training one or more care plan generation machine learning models to generate recommended care plans based on the summarized clinical data and predicted risk measures.
